(19) United States
(12) Patent Application Publication
(10) Pub. No.: US 2006/0078944 A1
(43) Pub. Date: Apr. 13, 2006

(54) Methods and reagents relating to inflammation and apoptosis

(76) Inventors: Jun Kuai, Cambridge, MA (US); Lih-Ling Lin, Concord, MA (US); Joseph L Wooters, Brighton, MA (US); Elliott Nickbarg, Belmont, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 10/523,328

(22) PCT Filed: Aug. 1, 2003
(86) PCT No.: PCT/US03/24340

§ 371 Date: Jun. 13, 2005

Related U.S. Application Data
(60) Provisional application No. 60/400,410, filed on Aug. 1, 2002.

(51) Int. Cl. G01N 33/53; C12P 21/02; C12N 9/12; C07K 14/535; C07K 14/715

(52) U.S. Cl. 435/7.1; 435/320.1; 435/325; 435/69.5; 530/350; 530/351; 435/194

(57) ABSTRACT

The present invention relates to, among other embodiments, protein complexes which include tumor necrosis factor alpha (TNF-α) and/or tumor necrosis factor alpha receptor (TNFR). Preferably, the complexes comprise at least one polypeptide selected from the group consisting of: NF-κB activating kinase (NAK), RasGAP3, TRCP1, and TRCP2. The present invention further provides assays of identifying a compound for modulating the stability and activity of the complex. Also provided are methods of modulating apoptosis and inflammation, as well as treating TNF-α related diseases.

### CROSS-REFERENCE TO RELATED APPLICATIONS

This application claims the benefit of priority of U.S. Provisional Application No. 60/400410, filed Aug. 1, 2002, the specification of which is incorporated by reference herein in its entirety.

### BACKGROUND

Tumor necrosis factor α (TNF-α) is a pro-inflammatory cytokine produced primarily by macrophages. The pleiotropic actions of TNF-α include inflammation, cell proliferation, differentiation, and apoptosis (Tracey et al., (1993) Annu. Rev. Cell Biol. 9:317-313; Baud et al., (2001) Trends in Cell Biol. 11:372-377). These actions are initiated by the binding of TNF-α to its receptors (i.e., TNFRs) that are expressed on most kinds of cells (Baglioni et al., 1985; Beutler et al., 1985; Kull et al., 1985; Tsujimoto et al., 1985; Aggarwal et al., 1985; Israel et al., 1986). The receptors provide the intracellular signals for cell response to TNF-α (Engelmann et al., 1990a).

TNF-α and TNFR play a role in inflammatory response. On one hand, TNF-α stimulates immunity, conferring resistance to infectious agents and resistance to tumors (Vilcek, et al., (1991) J. Biol. Chem. 266:7313-7316). On the other hand, TNF-α is implicated in a number of autoimmune diseases such as rheumatoid arthritis, graft rejection, and graft-versus-host diseases (Beutler, et al., (1998) Blood Cells Mol. Dis. 24:216-230; Beutler, (1999) J. Rheumatol. 26(Suppl) 57:16-21).

TNF-α and TNFR play another role in apoptosis, or programmed cell death. Apoptosis is a physiologic process essential to the normal development and homeostasis of multicellular organisms (H. Steller, (1995) Science 267:1445-1449). Derangements of apoptosis contribute to the pathogenesis of several human diseases including cancer, neurodegenerative disorders, and acquired immune deficiency syndrome (C. B. Thompson, (1995) Science 267:1456-1462).

The effects of TNF-α ligands and receptors are varied and influence numerous functions, both normal and abnormal, in the biological processes of the mammalian system. There is a clear need, therefore, for identification and characterization of protein complexes comprising such receptors and ligands, which influence biological activity, both normally and in disease states.

### BRIEF SUMMARY

In certain aspects, the invention provides an isolated, purified, or recombinant protein complex comprising a TNF-α polypeptide, a TNFR polypeptide and at least one polypeptide selected from the group consisting of: NF-κB activating kinase (NAK), RasGAP3, TRCP1, and TRCP2. NAK is also known as TBK1 (TANK-binding kinase) or T2K (TRAF2-associated kinase). In certain embodiments, the isolated, purified, or recombinant protein complex further comprises at least one polypeptide selected from the group consisting of: TRADD, TRAF2, and TRAP2.

In certain aspects, the invention provides an isolated, purified, or recombinant protein complex comprising a TNFR polypeptide and at least one polypeptide selected from the group consisting of: NF-κB activating kinase (NAK), RasGAP3, TRCP1, and TRCP2. In certain embodiments, the isolated, purified, or recombinant protein complex further comprises at least one polypeptide selected from the group consisting of: TNF-α, TRADD, TRAF2, and TRAP2.

In a specific embodiment, the protein complex of the present invention comprises a TNF-α polypeptide, a TNFR polypeptide, a NAK polypeptide, a RasGAP3 polypeptide, a TRCP1 polypeptide, a TRCP2 polypeptide, a TRADD polypeptide, a TRAF2 polypeptide, and a TRAP2 polypeptide. For example, the TNFR polypeptide of the complex can be a TNFR1 or TNFR2 polypeptide.

In certain embodiments, one or more of the polypeptides of a complex of the invention is a variant, such as a fragment, a fusion protein, a labeled protein, etc., and preferably the variant is a functional variant.

In further aspects, the invention provides host cells comprising one or more recombinant nucleic acids encoding one or more polypeptide constituents of a complex disclosed herein. In certain embodiments, the host cells comprise a first nucleic acid, a second nucleic acid and a third nucleic acid, wherein the first nucleic acid comprises a recombinant nucleic acid encoding a TNFR polypeptide, the second nucleic acid comprises a recombinant nucleic acid encoding a TNF-α polypeptide, and the third nucleic acid comprises a recombinant nucleic acid encoding a polypeptide selected from the group consisting of: NAK, RasGAP3, TRCP1, and TRCP2. In certain embodiments, the host cells comprise a first nucleic acid and a second nucleic acid, wherein the first nucleic acid comprises a recombinant nucleic acid encoding a TNFR polypeptide, and wherein the second nucleic acid comprises a recombinant nucleic acid encoding a polypeptide selected from the group consisting of: NAK, RasGAP3, TRCP1, and TRCP2.

In certain aspects, the invention provides assays for identifying test compounds that inhibit or potentiate the stability of a protein complex disclosed herein. In certain embodiments, an assay comprises: forming a reaction mixture including TNF-α, TNFR, and at least one polypeptide selected from the group consisting of: NAK, RasGAP3, TRCP1, and TRCP2, and a test compound; and detecting the presence of TNF-α or TNFR in the complex. A change in the presence of TNF-α or TNFR in the complex in the presence of the test compound, relative to the presence of TNF-α or TNFR in the complex in the absence of the test compound, indicates that said test compound potentiates or inhibits the stability of said complex.

In certain embodiments, an assay of the invention comprises the following two steps: (i) forming a reaction mixture including TNF-α, TNFR, at least one polypeptide selected from the group consisting of: NAK, RasGAP3, TRCP1, and TRCP2, and a test compound; and (ii) detecting the association between the TNF-α or TNFR and a polypeptide selected from the group consisting of: NAK, RasGAP3, TRCP1, and TRCP2. A change in the association between TNF-α or TNFR and a polypeptide selected from the group consisting of: NAK, RasGAP3, TRCP1, and TRCP2 in the presence of the test compound, relative to the association between TNF-α or TNFR and a polypeptide selected from the group consisting of: NAK, RasGAP3, TRCP1, and TRCP2 in the absence of the test compound, indicates that said test compound potentiates or inhibits the stability of said complex. In certain embodiments, an assay of the invention comprises the following two steps: (i) forming a reaction mixture including TNFR, at least one polypeptide selected from the group consisting of: NAK, RasGAP3, TRCP1, and TRCP2, and a test compound; and (ii) detecting the association between the TNFR and a polypeptide selected from the group consisting of: NAK, RasGAP3, TRCP1, and TRCP2. Optionally, the reaction mixture may contain TNF-α. A change in the association between TNFR and a polypeptide selected from the group consisting of: NAK, RasGAP3, TRCP1, and TRCP2 in the presence of the test compound, relative to the association between TNFR and a polypeptide selected from the group consisting of: NAK, RasGAP3, TRCP1, and TRCP2 in the absence of the test compound, indicates that said test compound potentiates or inhibits the stability of said complex.

In further embodiments, test compound screening assays comprise: (i) forming a protein complex comprising TNFR and a polypeptide selected from the group consisting of: NAK, RasGAP3, TRCP1, and TRCP2; (ii) contacting the protein complex with a test compound; and (iii) determining the effect of the test compound for one or more activities. The protein complex may also comprise TNF-α. Such activities are selected from the group comprising a change in the level of the protein complex; a change in the level of the TNFR or TNF-α polypeptide in the complex; a change in the signaling enzymatic activity of the complex; or a change in the interaction between the TNFR or TNF-α polypeptide and the polypeptide selected from the group consisting of: NAK, RasGAP3, TRCP1, and TRCP2.

In further embodiments, the invention provides screening assays comprising: providing a two-hybrid assay system including a first fusion protein (e.g., comprising a TNFR polypeptide portion), and a second fusion protein (e.g., comprising a portion of a polypeptide selected from the group consisting of: NAK, RasGAP3, TRCP1, and TRCP2), under conditions wherein said two hybrid assay is sensitive to interactions between the first fusion protein (e.g., comprising a TNFR polypeptide) and the second fusion protein (e.g., comprising a polypeptide selected from the group consisting of: NAK, RasGAP3, TRCP1, and TRCP2); measuring a level of interactions between said fusion proteins in the presence and in the absence of a test compound; and comparing the level of interaction of said fusion proteins. A decrease in the level of interaction is indicative of a compound that will inhibit the interaction between the fusion proteins (e.g., between a TNFR polypeptide and a polypeptide selected from the group consisting of: NAK, RasGAP3, TRCP1, and TRCP2). This assay of the invention can be used for any two proteins of the complex (e.g., the complex consisting of: TNF-α, TNFR, NAK, RasGAP3, TRCP1, and TRCP2).

In further aspects, the invention provides antibodies, or fragments thereof, specifically immunoreactive with an epitope of a polypeptide of a complex disclosed herein, such as a polypeptide selected from the group consisting of: TNF-α, TNFR, NAK, RasGAP3, TRCP1, and TRCP2. Preferably the antibody disrupts formation of an interaction between TNF-α or TNFR and a polypeptide selected from the group consisting of: NAK, RasGAP3, TRCP1, and TRCP2.

In certain aspects, the invention provides methods for modulating, in a cell, a protein complex comprising a first protein, a second protein and a third protein, wherein said first protein is TNF-α, said second protein is TNFR and said third protein is selected from the group consisting of: NAK, RasGAP3, TRCP1, and TRCP2. In certain aspects, the invention provides methods for modulating, in a cell, a protein complex comprising a first protein and a second protein, wherein said first protein is TNF-α or TNFR and said second protein is selected from the group consisting of: NAK, RasGAP3, TRCP1, and TRCP2. The method comprises administering to said cell a compound capable of modulating said protein complex.

Further aspects of the invention relate to methods of producing a functional complex comprising: transfecting a cell with a polynucleotide encoding a polypeptide selected from the group consisting of: NAK, RasGAP3, TRCP1, and TRCP2; contacting said cell with a TNF-α or TNFR polypeptide; thereby forming a protein complex.

A further aspect of the invention relates to methods for treating a TNFα-related disorder, by administering an effective amount of an compound that inhibits the interaction of TNF-α or TNFR with a polypeptide selected from the group consisting of: NAK, RasGAP3, TRCP1, and TRCP2.

Other aspects of the invention relate to methods of identifying a test compound that is a candidate modulator of inflammation or apoptosis. Such methods comprise: (i) forming a mixture comprising a TRCP1 or TRCP2 polypeptide or a variant polypeptide thereof, and a test compound; and (ii) measuring the interaction between the TRCP1 or TRCP2 polypeptide or the variant and the test compound; wherein a test compound that interacts with the TRCP1 or TRCP2 polypeptide or the functional variant thereof is a candidate modulator of inflammation or apoptosis. In such methods, the first step (i) may comprise forming the mixture in vitro, or comprise contacting a cell expressing a TRCP1 or TRCP2 polypeptide or a variant thereof, with the test compound.

Accordingly, a further aspect of the invention relates to methods of treating a TNF-α-related disease which includes an inflammatory or apoptotic component, by administering an effective amount of a therapeutic composition that modulates TRCP1 or TRCP2.

The embodiments and practices of the present invention, other embodiments, and their features and characteristics, will be apparent from the description, figures and claims that follow, with all of the claims hereby being incorporated by this reference into this Summary.

### BRIEF DESCRIPTION OF THE DRAWINGS

FIG. 1 illustrates formation of a TNF-α receptor complex induced by the addition of TNF-α ligand. A. Proteins in the TNF-α receptor complex are separated by SDS-PAGE gel and visualized by silver staining. B. The presence of a protein, TRADD, in the TNF-α receptor complex is confirmed by western blotting.

FIG. 2 shows the amino acid sequence (SEQ ID No: 1) of tumor necrosis factor α (TNF-α).

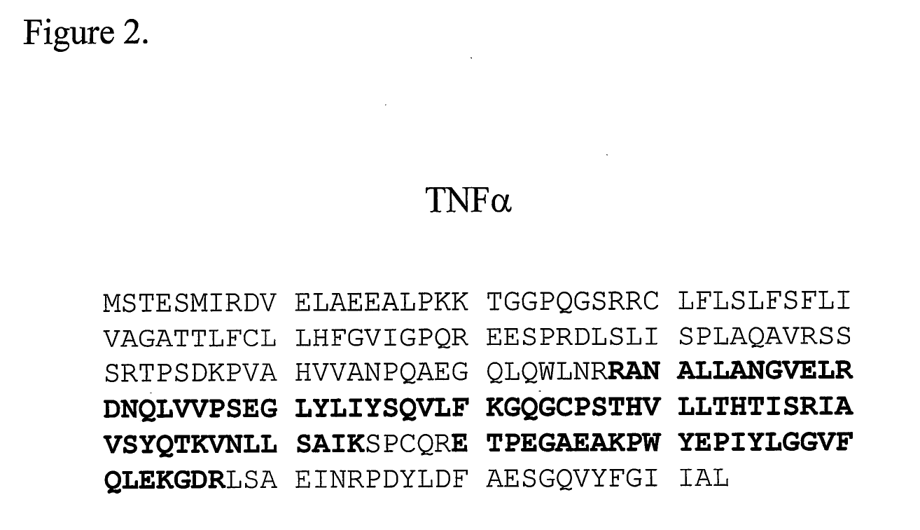

FIG. 3 shows the cDNA sequence (SEQ ID No: 2) encoding TNF-α.

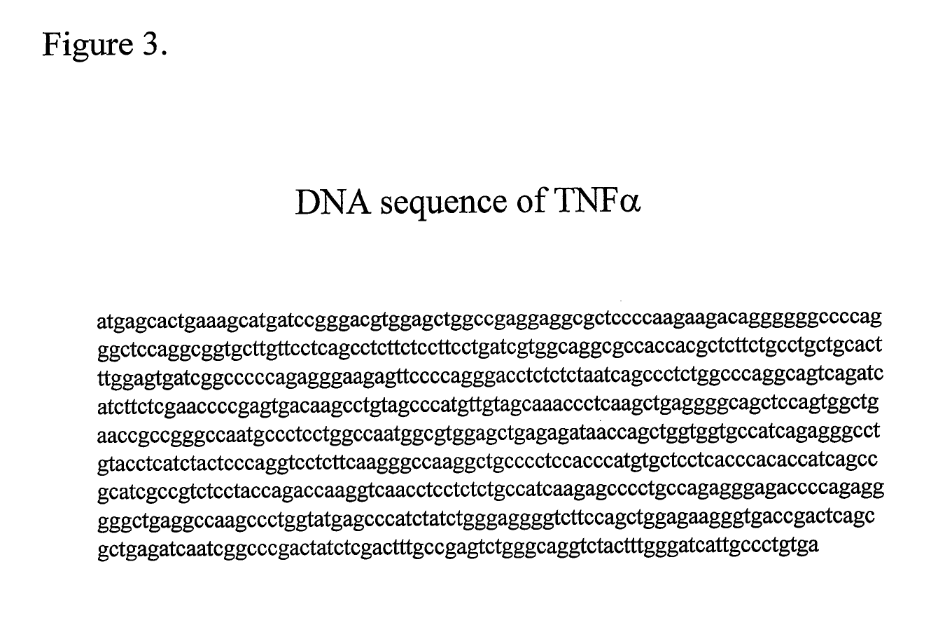

FIG. 4 shows the amino acid sequence (SEQ ID No: 3) of TNF-α receptor 1 (TNFR1).

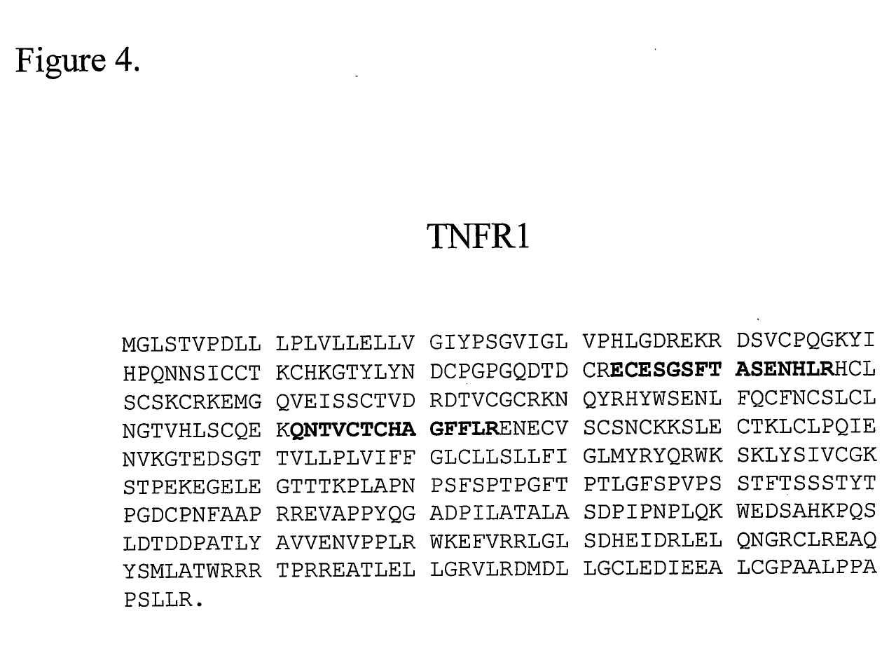

FIG. 5 shows the cDNA sequence (SEQ ID No: 4) encoding TNFR1.

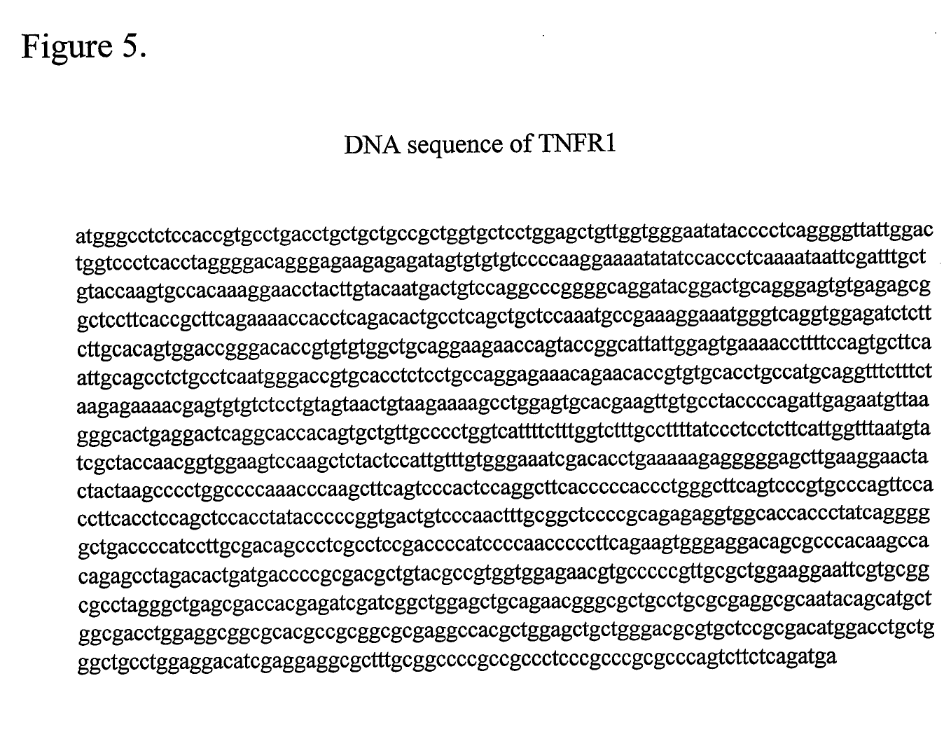

FIG. 6 shows the amino acid sequence (SEQ ID No: 5) of TNF-α receptor 2 (TNFR2).

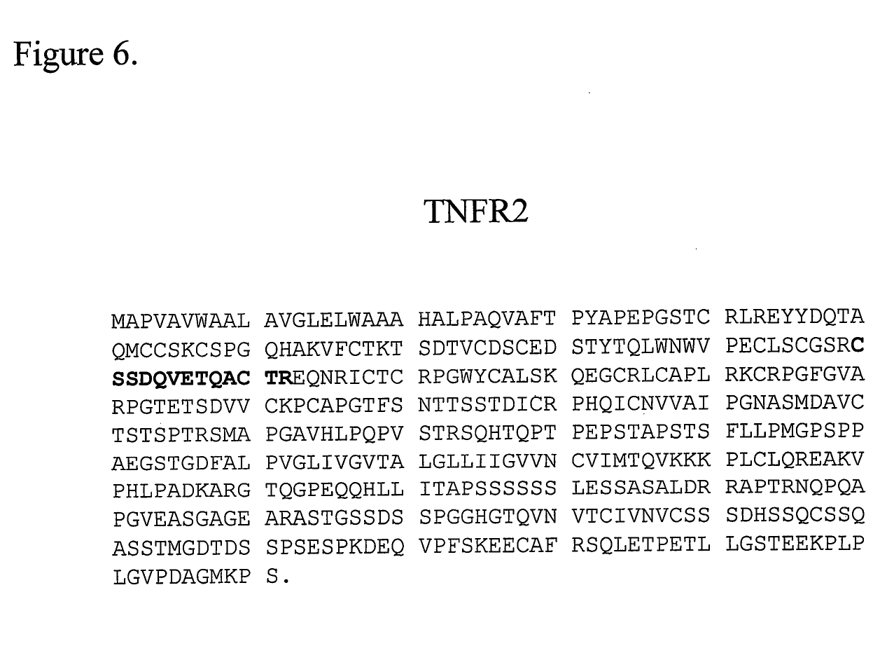

FIG. 7 shows the cDNA sequence (SEQ ID No: 6) encoding TNFR2.

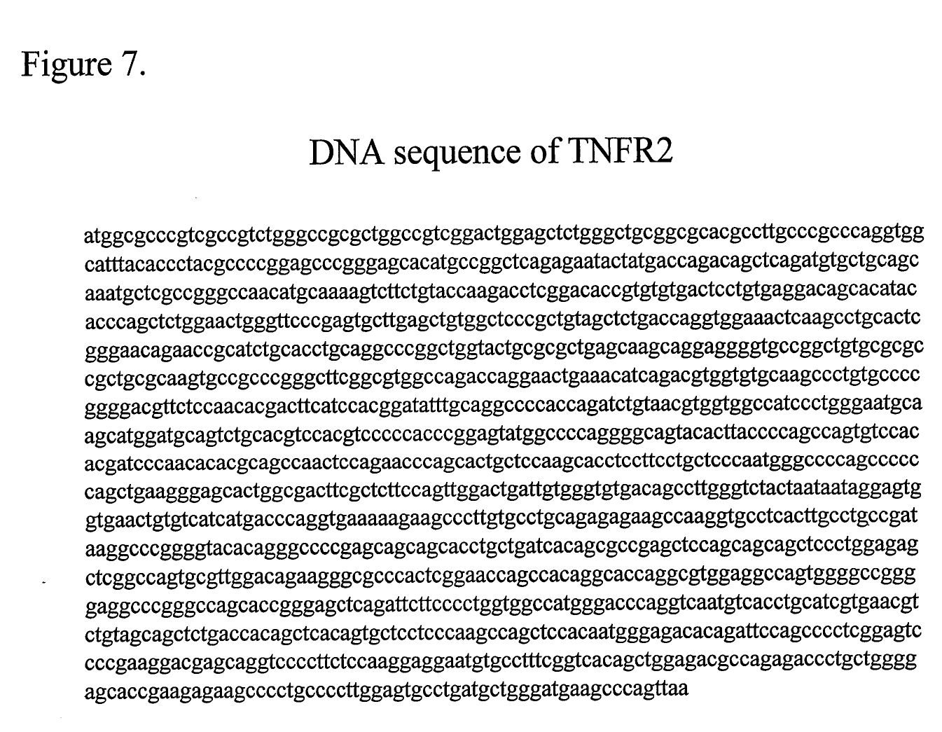

FIG. 8 shows the amino acid sequence (SEQ ID No: 7) of TRADD, a protein identified in the TNF-α receptor complex.

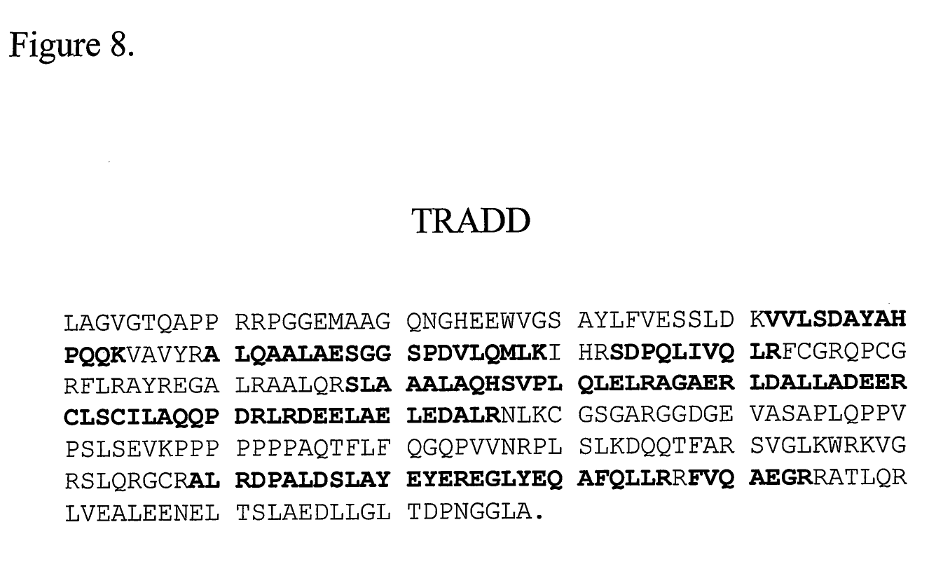

FIG. 9 shows the cDNA sequence (SEQ ID No: 8) encoding TRADD.

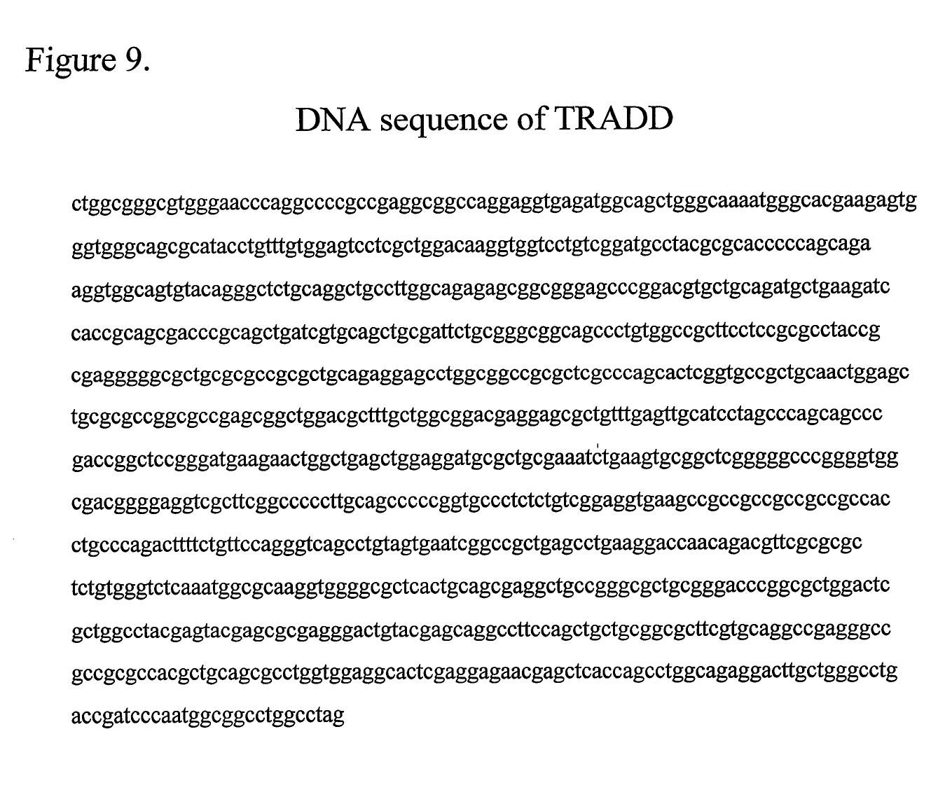

FIG. 10 shows the amino acid sequence(SEQ ID No: 9) of TRAF2, a protein identified in the TNF-α receptor complex.

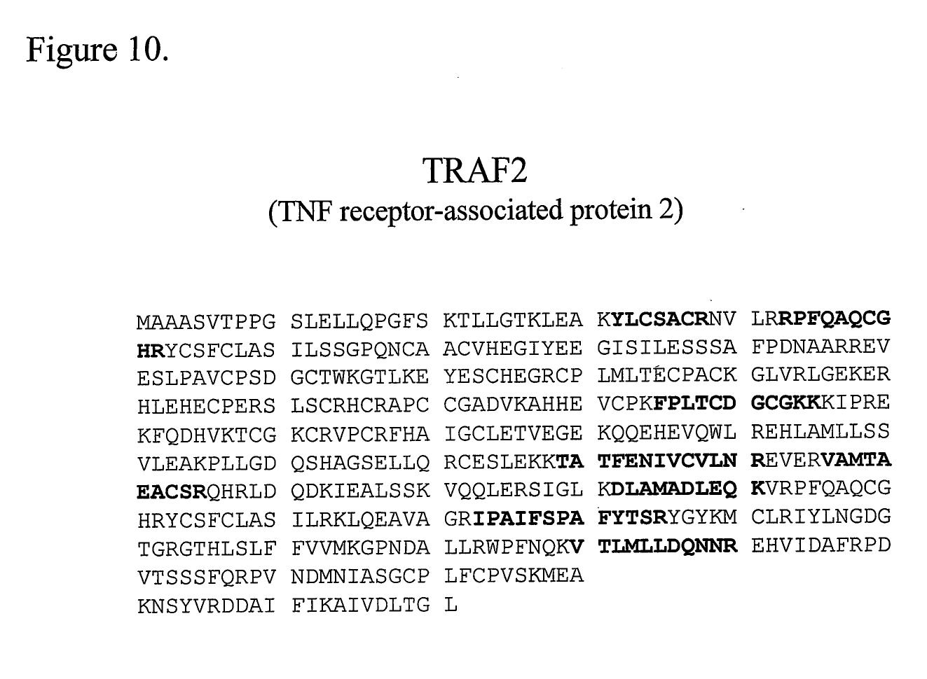

FIG. 11 shows the cDNA sequence (SEQ ID No: 10) encoding TRAF2.

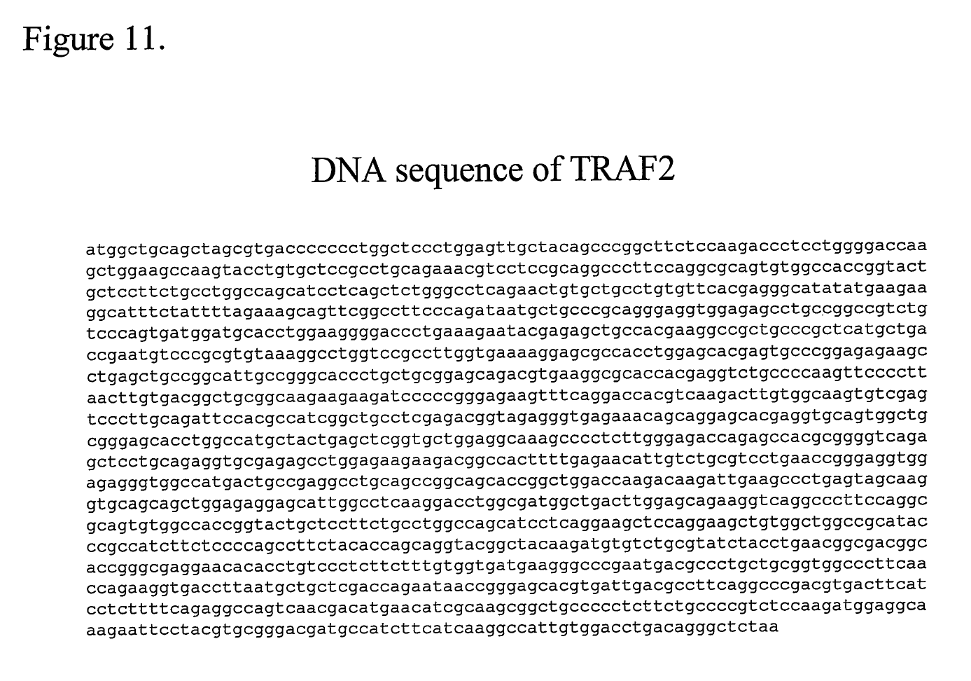

FIG. 12 shows the amino acid sequence (SEQ ID No: 11) of TRAP2, a protein identified in the TNF-α receptor complex.

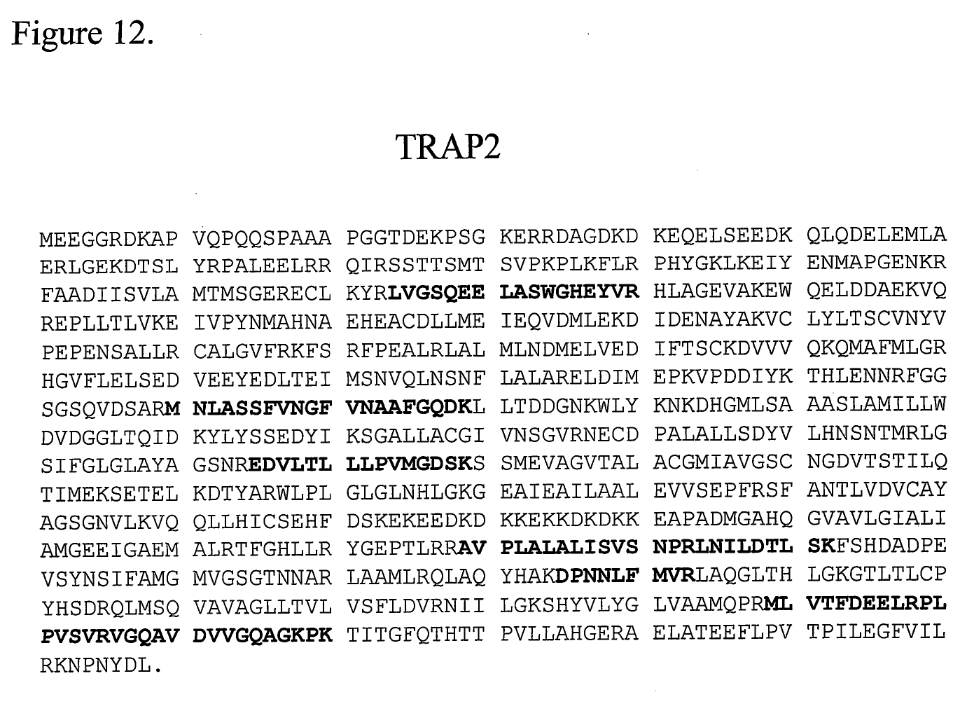

FIG. 13 shows the cDNA sequence (SEQ ID No: 12) encoding TRAP2.

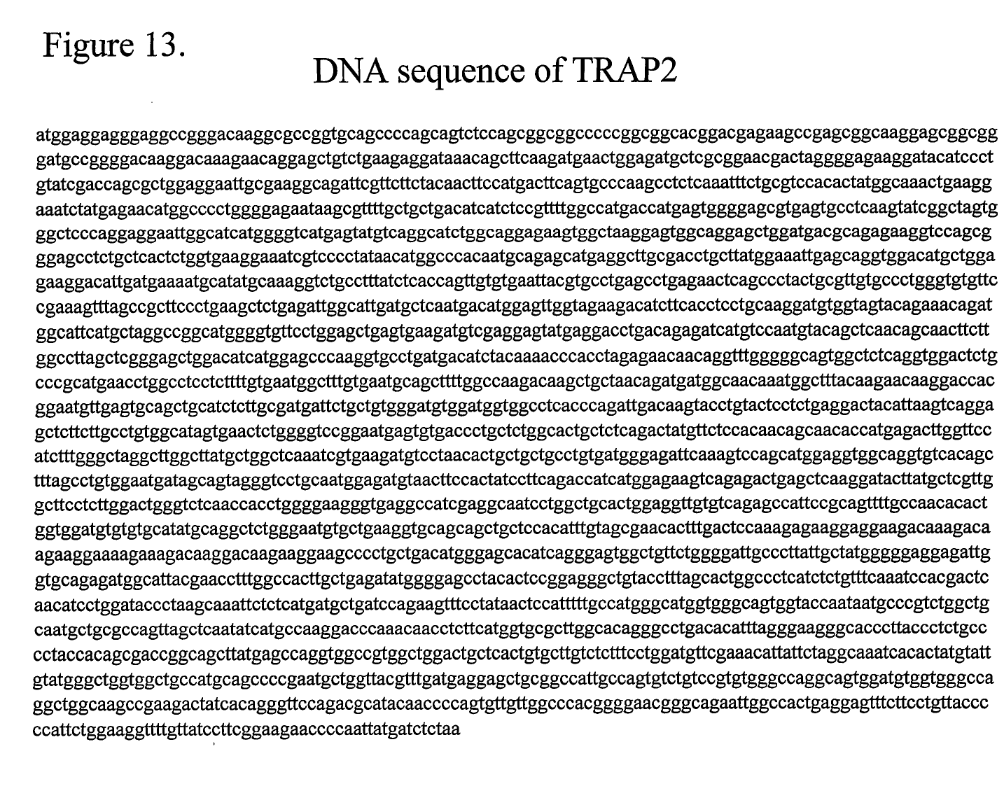

FIG. 14 shows the amino acid sequence (SEQ ID No: 13) of NAK (also referred to as TBK or T2K), a protein identified in the TNF-α receptor complex.

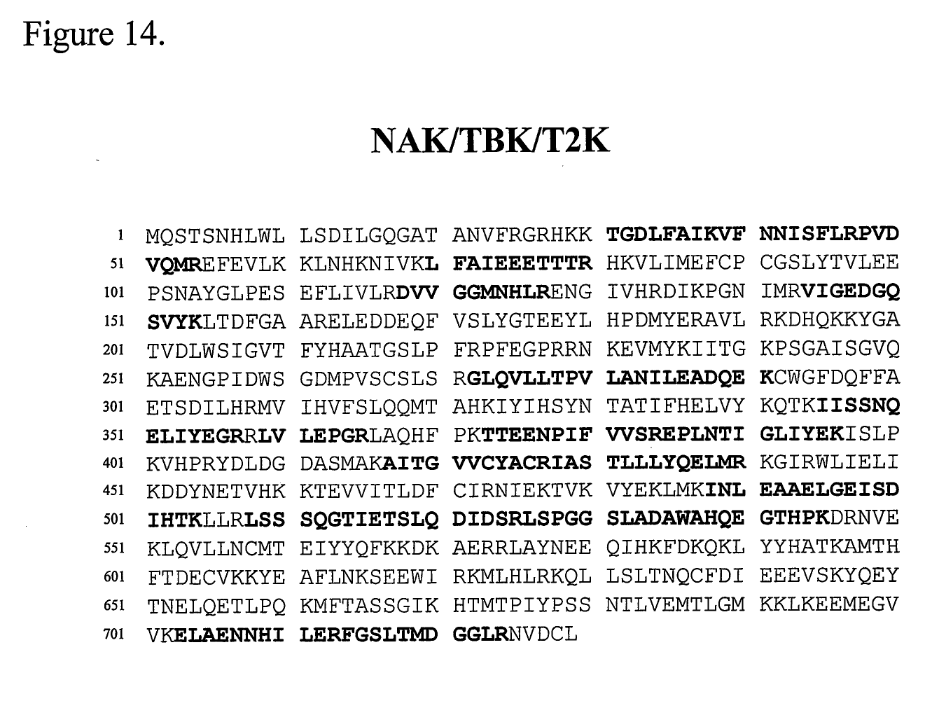

FIG. 15 shows the cDNA sequence (SEQ ID No: 14) encoding NAK (also referred to as TBK or T2K).

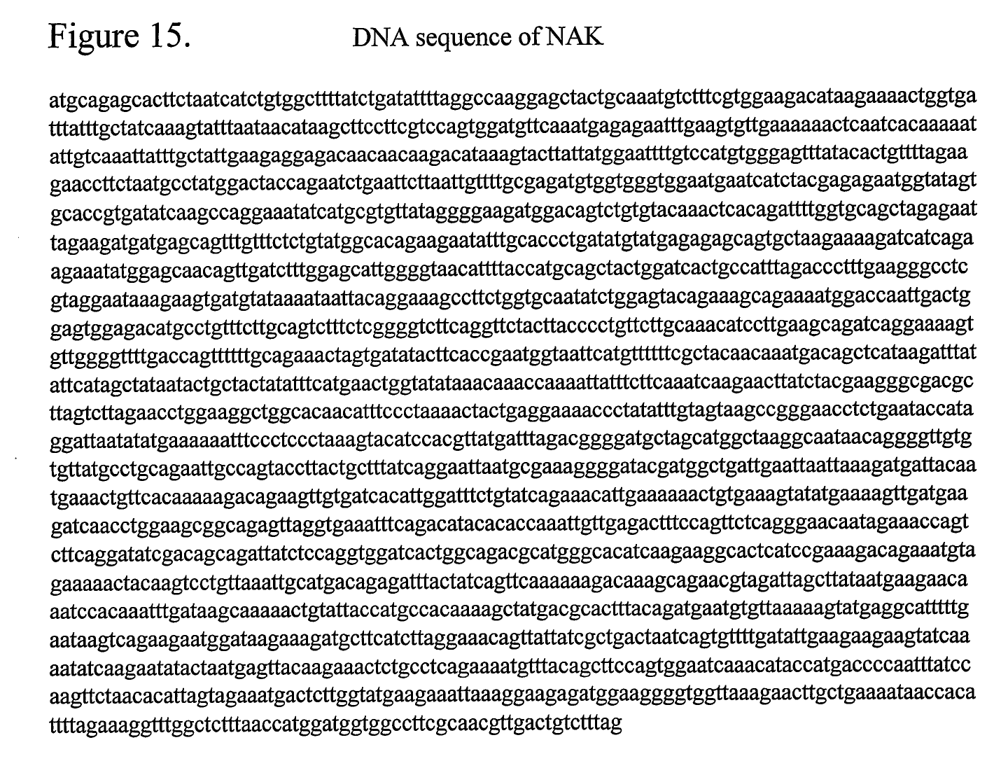

FIG. 16 shows the amino acid sequence (SEQ ID No: 15) of RasGAP3, a protein identified in the TNF-α receptor complex.

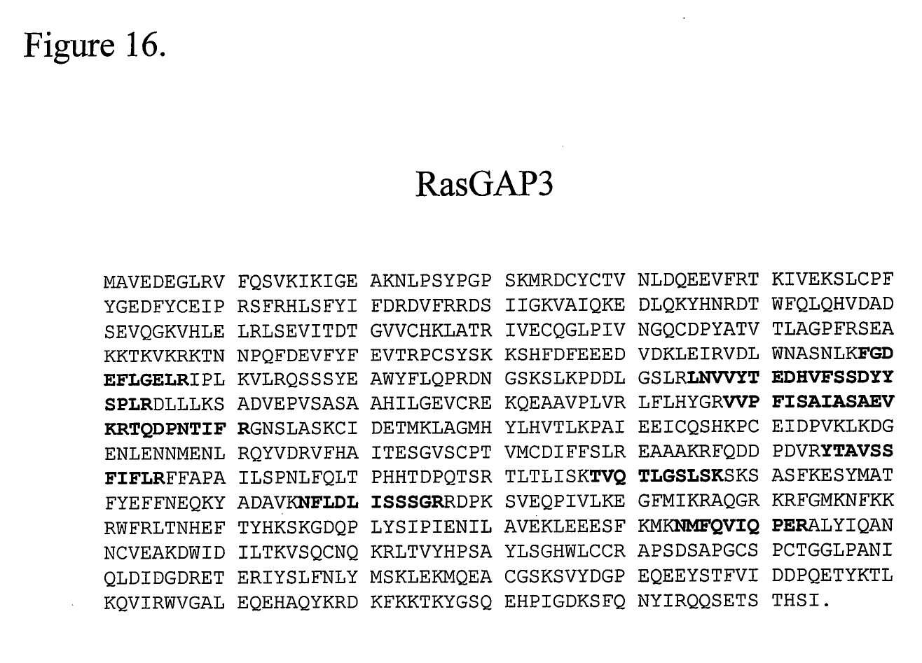

FIG. 17 shows the cDNA sequence (SEQ ID No: 16) encoding RasGAP3.

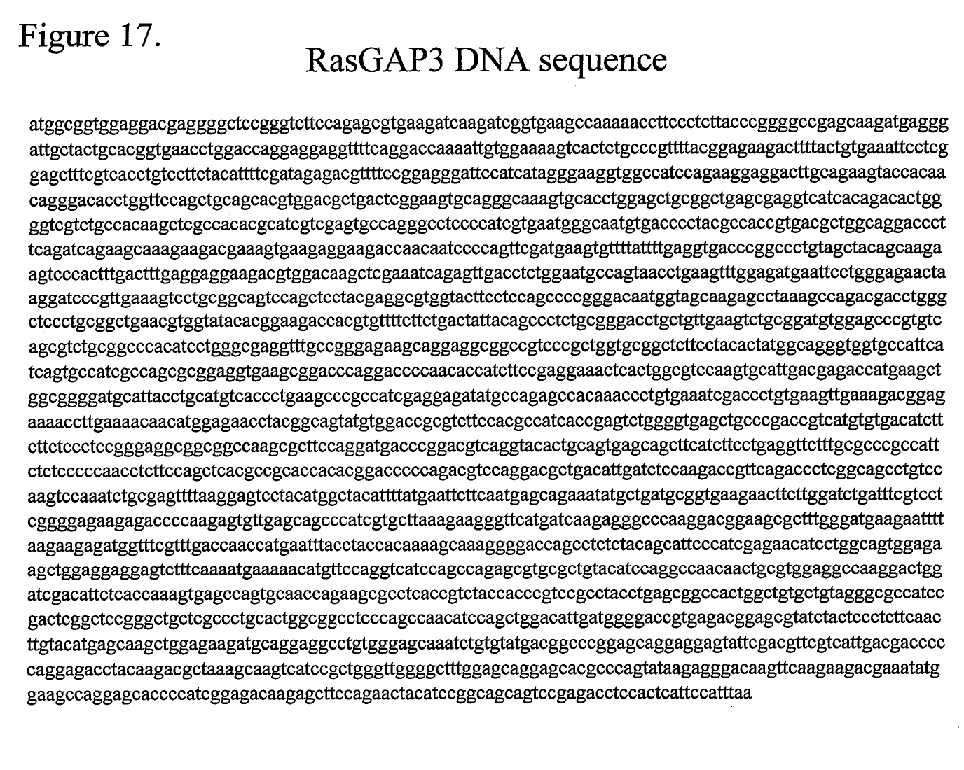

FIG. 18 shows the amino acid sequence (SEQ ID No: 17) of TRCP1 (also referred to as KIAA0143), a protein identified in the TNF-α receptor complex.

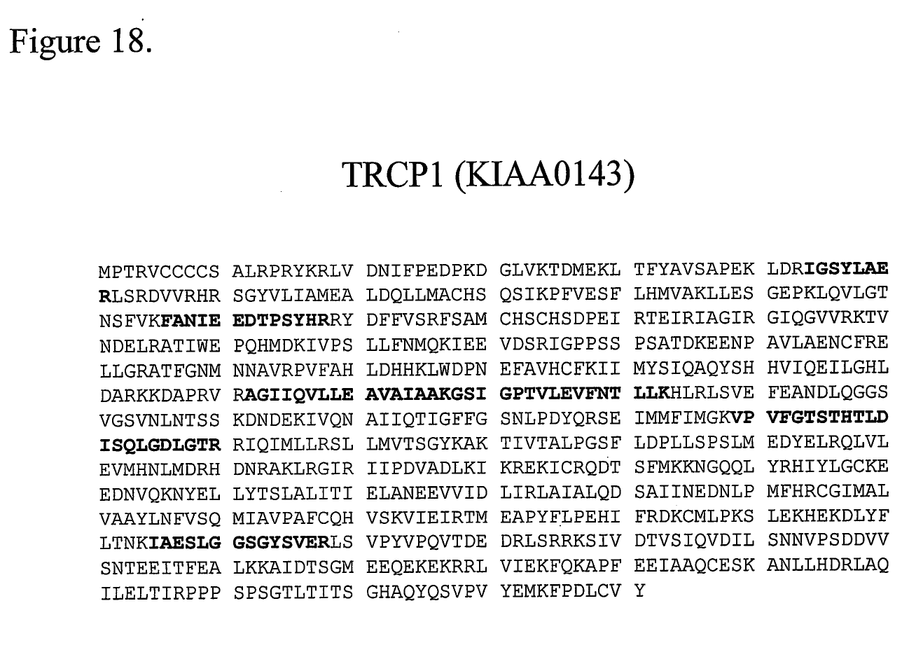

FIG. 19 shows the cDNA sequence (SEQ ID No: 18) encoding TRCP1 (also referred to as KIAA0143).

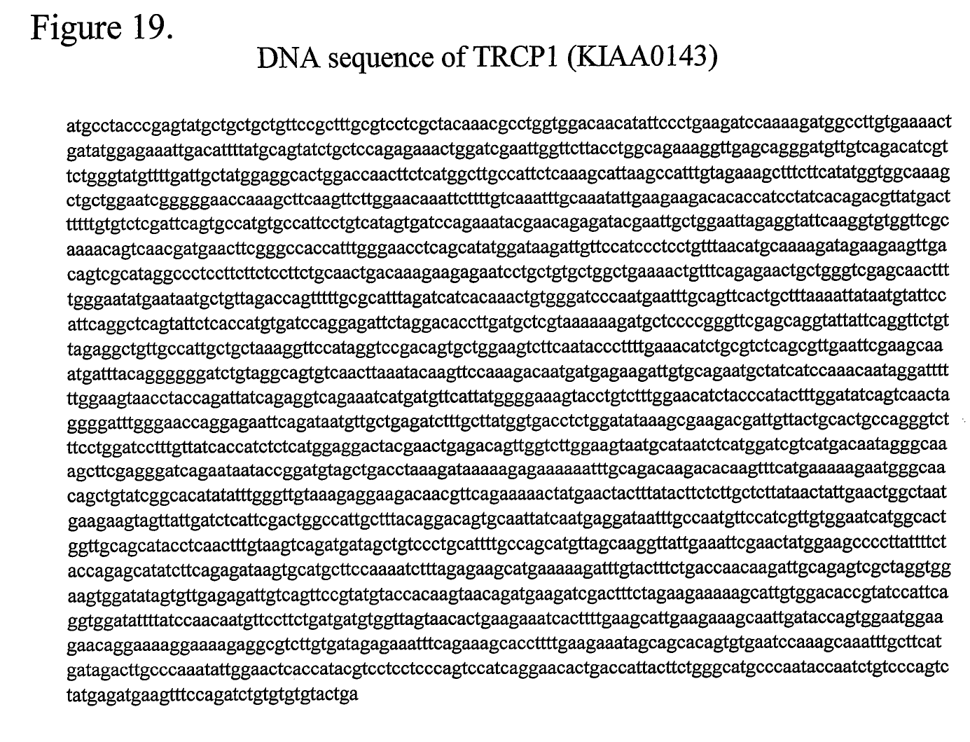

FIG. 20 shows the amino acid sequence (SEQ ID No: 19) of IRCP2 (similar to as FLJ20758), a protein identified in the TNF-α receptor complex.

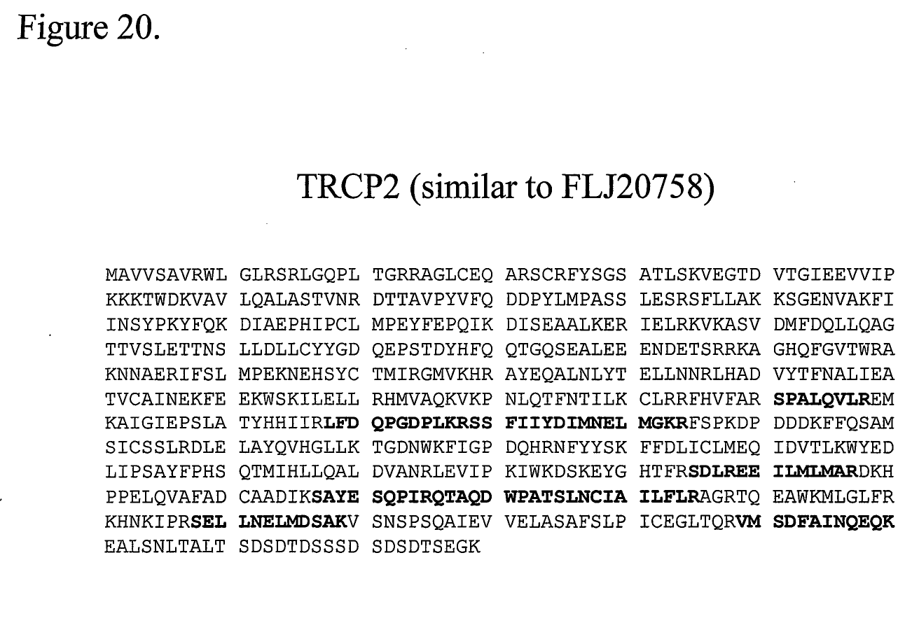

FIG. 21 shows the cDNA sequence (SEQ ID No: 20) encoding TRCP2.

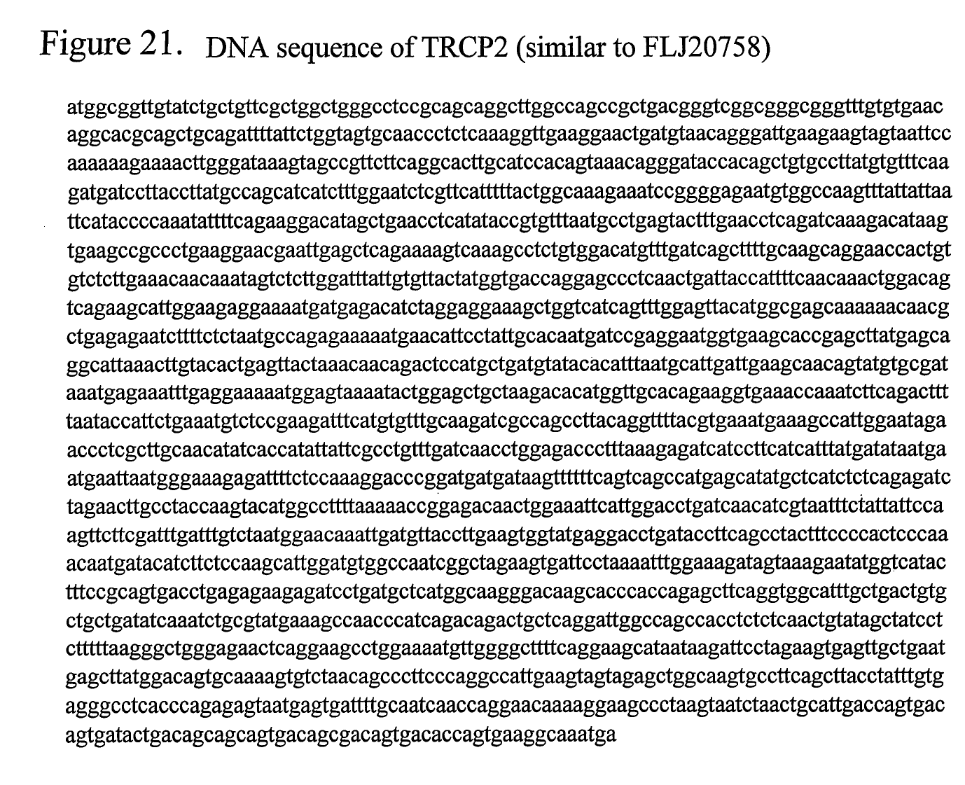

FIG. 22 illustrates TNF-α dependent recruitment of NAK, TRAF2, and TRADD on TNFR1.

### DETAILED DESCRIPTION

I. Overview

Tumor necrosis factor a (TNF-α) is a cytokine produced primarily by lymphocytes, macrophages and several other cell types involved in a broad range of cellular responses including autoimmune responses, cell proliferation, differentiation and apoptosis. TNF-α belongs to a family of trimeric cytokines that bind their target receptors on the cell surface and bring about trimerization/aggregation of TNF receptors, such as TNFR1 and TNFR2 which are part of a larger TNF receptor superfamily. The interaction of TNF-α with its receptor(s) and the subsequent complex formation is involved in signaling responses within the cell, such responses including activation of a Caspase cascade leading to apoptosis or activation of the transcription factors AP-1 and NF-κB that in turn result in the transcriptional activation of genes involved in chronic and acute inflammatory responses. Also, some of the latter genes, primarily those dependent on NF-κB, function to suppress apoptosis in certain circumstances or cell types.

II. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “binding” refers to a direct or indirect association between two molecules. Direct associations may include, for example, covalent, electrostatic, hydrophobic, ionic and/or hydrogen-bond interactions under physiological conditions. Indirect associations include, for example, two proteins that are part of a complex but do not have any direct interactions.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric protein” or “fusion protein” is a fusion of a first amino acid sequence encoding a polypeptide with a second amino acid sequence, wherein the first and second amino acid sequences do not occur naturally as part of a singly polypeptide chain.

The phrase “conservative amino acid substitution” refers to grouping of amino acids on the basis of certain common properties. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer., Principles of Protein Structure, Springer-Verlag). Examples of amino acid groups defined in this manner include:
(i) a charged group, consisting of: Glu and Asp, Lys, Arg and His,
(ii) a positively-charged group, consisting of: Lys, Arg and His,
(iii) a negatively-charged group, consisting of: Glu and Asp,
(iv) an aromatic group, consisting of: Phe, Tyr and Trp,
(v) a nitrogen ring group, consisting of: His and Trp,
(vi) a large aliphatic nonpolar group, consisting of: Val, Leu and Ile,
(vii) a slightly-polar group, consisting of: Met and Cys,
(viii) a small-residue group, consisting of: Ser, Thr, Asp, Asn, Gly, Ala, Glu, Gln and Pro,
(ix) an aliphatic group consisting of: Val, Leu, Ile, Met and Cys, and
(x) a small hydroxyl group consisting of: Ser and Thr.

In addition to the groups presented above, each amino acid residue may form its own group, and the group formed by an individual amino acid may be referred to simply by the one and/or three letter abbreviation for that amino acid commonly used in the art.

The terms “compound”, “test compound,” and “molecule” are used herein interchangeably and are meant to include, but are not limited to, peptides, nucleic acids, carbohydrates, small organic molecules, natural product extract libraries, and any other molecules (including, but not limited to, chemicals, metals, and organometallic compounds).

A “conserved residue” is an amino acid that is relatively invariant across a range of similar proteins. Often conserved residues will vary only by being replaced with a similar amino acid, as described above for “conservative amino acid substitution.”

The term “domain” as used herein refers to a region of a protein that comprises a particular structure and/or performs a particular function.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, preferably less than 25% identity with a sequence of the present invention. In comparing two sequences, the absence of residues (amino acids or nucleic acids) or presence of extra residues also decreases the identity and homology/similarity.

The term “homology” describes a mathematically based comparison of sequence similarities which is used to identify genes or proteins with similar functions or motifs. The nucleic acid and protein sequences of the present invention may be used as a “query sequence” to perform a search against public databases to, for example, identify other family members, related sequences or homologs. Such searches can be performed using the NBLAST and XBLAST programs (version 2.0) of Altschul, et al. (1990) J Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used. See http://www.ncbi.nlm.nih.gov.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

The term “including” is used herein to mean, and is used interchangeably with, the phrase “including but not limited to”.

The term “isolated”, as used herein with reference to the subject proteins and protein complexes, refers to a preparation of protein or protein complex that is essentially free from contaminating proteins that normally would be present with the protein or complex, e.g., in the cellular milieu in which the protein or complex is found endogenously. Thus, an isolated protein complex is isolated from cellular components that normally would “contaminate” or interfere with the study of the complex in isolation, for instance while screening for modulators thereof. It is to be understood, however, that such an “isolated” complex may incorporate other proteins the modulation of which, by the subject protein or protein complex, is being investigated.

The term “isolated” as also used herein with respect to nucleic acids, such as DNA or RNA, refers to molecules in a form which does not occur in nature. Moreover, an “isolated nucleic acid” is meant to include nucleic acid fragments which are not naturally occurring as fragments and would not be found in the natural state.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

The term “or” is used herein to mean, and is used interchangeably with, the term “and/or”, unless context clearly indicates otherwise.

The terms “proteins,” and “polypeptides” are used interchangeably herein.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene as described in the attached examples. By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 85% by weight, more preferably 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above.

The term “recombinant nucleic acid” includes any nucleic acid comprising at least two sequences which are not present together in nature. A recombinant nucleic acid may be generated in vitro, for example by using the methods of molecular biology, or in vivo, for example by insertion of a nucleic acid at a novel chromosomal location by homologous or non-homologous recombination.

The term “recombinant protein” refers to a protein of the present invention which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

“Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures comprising arrays of small molecules, often fungal, bacterial, or algal extracts, which can be screened with any of the assays of the invention.

As used herein, the term “specifically hybridizes” refers to the ability of a nucleic acid probe/primer of the invention to hybridize to at least 12, 15, 20, 25, 30, 35, 40, 45, 50 or 100 consecutive nucleotides of a target sequence, or a sequence complementary thereto, or naturally occurring mutants thereof, such that it has less than 15%, preferably less than 10%, and more preferably less than 5% background hybridization to a cellular nucleic acid (e.g., mRNA or genomic DNA) other than the target gene. A variety of hybridization conditions may be used to detect specific hybridization, and the stringency is determined primarily by the wash stage of the hybridization assay. Generally high temperatures and low salt concentrations give high stringency, while low temperatures and high salt concentrations give low stringency. Low stringency hybridization is achieved by washing in, for example, about 2.0×SSC at 50° C., and high stringency is achieved with about 0.2×SSC at 50° C. Further descriptions of stringency are provided below.

As applied to polypeptides, “substantial sequence identity” means that two peptide sequences, when optimally aligned, such as by the programs GAP or BESTFIT using default gap which share at least 90 percent sequence identity, preferably at least 95 percent sequence identity, more preferably at least 99 percent sequence identity or more. Preferably, residue positions which are not identical differ by conservative amino acid substitutions. For example, the substitution of amino acids having similar chemical properties such as charge or polarity are not likely to effect the properties of a protein. Examples include glutamine for asparagine or glutamic acid for aspartic acid.

The term “tumor necrosis factor alpha receptor” or “TNFR” includes TNFR1, TNFR2, and functional fragments and analogs thereof. The term TNFR also includes any polypeptide that binds to TNF-α and transduces such binding so as to affect an intracellular signaling pathway.

A “variant” of a polypeptide, such as, for example, a variant of a TNF-α, a TNFR, a TRCP1 or a TRCP2 includes chimeric proteins, fusion proteins, mutant proteins, proteins having similar but non-identical sequences, protein fragments, mimetics, etc, so long as the variant has at least a portion of an amino acid sequence of a native protein, or at least a portion of an amino acid sequence of substantial sequence identity to the native protein. A “functional variant” includes a variant that retains at least one function of the native protein. As used herein, the term “tumor necrosis factor alpha” or “TNF-α” includes functional variants of TNF-α.

III. Protein Complexes

In certain embodiments, the present invention relates to the discovery of novel protein complexes. In certain embodiments, protein complexes of the invention comprise a TNF-α polypeptide, a TNFR polypeptide and at least one polypeptide selected from the group consisting of: NAK, RasGAP3, TRCP1, and TRCP2. Optionally, such protein complexes further comprise at least one polypeptide selected from the group consisting of: TRADD, TRAF2, and TRAP2 or a polypeptide known to form a complex with TNF-α or TNFR, such as TRAF1, RIP, RAIDD, or FADD.

In a specific embodiment, protein complexes of the invention comprise polypeptides of TNF-α, TNFR1, NAK, TRAF2, and TRADD.

In another specific embodiment, the protein complex of the present invention comprises a TNF-α polypeptide, a TNFR polypeptide, a NAK polypeptide, a RasGAP3 polypeptide, a TRCP1 polypeptide, a TRCP2 polypeptide, a TRADD polypeptide, a TRAF2 polypeptide, and a TRAP2 polypeptide. For example, the TNFR polypeptide of the complex can be a TNFR1 or TNFR2 polypeptide.

In further embodiments, protein complexes of the invention comprise a TNFR and at least one polypeptide selected from the group consisting of: NAK, RasGAP3, TRCP1, and TRCP2. Optionally, such protein complexes further comprise at least one polypeptide selected from the group consisting of: TRADD, TRAF2, and TRAP2 or a polypeptide known to form a complex with TNFR, such as TRAF1, RIP, RAIDD, or FADD.

The present invention also contemplates protein complexes comprising any two polypeptides selected from the group consisting of: TNF-α, TNFR, NAK, RasGAP3, TRCP1, and TRCP2. Optionally, such protein complexes further comprise at least one polypeptide selected from the group consisting of: TRADD, TRAF2, and TRAP2 or a polypeptide known to form a complex with TNFR, such as TRAF1, RIP, RAIDD, or FADD.

Optionally, one or more of the polypeptides of a complex is a variant of that polypeptide, and preferably a functional variant of that polypeptide. For example, in one embodiment, protein complexes of the invention comprise TNF-α and NAK wherein either TNF-α and/or NAK may be represented by a variant of TNF-α and/or NAK.

Complexes of the invention may be obtained in essential form, such as, for example, as an isolated complex, a recombinant complex, a purified complex, etc. In an embodiment, the invention provides a protein complex prepared, for example, by extraction from a cell that comprises the complex. Extraction from a cell may be accomplished by any of the many methods known in the art. For example, a complex may be extracted from the cell by a series of traditional protein purification steps, such as centrifugation, gel filtration, ion exchange chromatography, etc., and it will generally be preferable to select purification steps and conditions that do not dissociate the complex. Extraction from a cell may also be achieved by affinity purification. For example, one or more of the proteins in the desired complex may be expressed as a fusion protein comprising an affinity purification tag, such as a hexahistidine tag, a glutathione-S-transferase (GST) tag, etc. The complex may then be purified by an appropriate affinity purification (e.g., contacting with a nickel or copper resin in the case of a hexahistidine tag, contacting with a glutathione resin in the case of a GST tag. In another preferred form, the invention provides a protein complex, for example, prepared by purifing recombinant polypeptides expressed in cells such as E. coli and reconstituting the complex in vitro. In certain embodiments, one or more of the constituent polypeptides of a complex is expressed from an endogenous gene of a cell. In certain embodiments, complexes are recombinant complexes wherein one or more of the constituent polypeptides is expressed from a recombinant nucleic acid.

In certain embodiments, the invention also includes labeled protein complexes, wherein at least one polypeptide of the complex is labeled. Most preferably, the label is a detectable label, selected from, but not limited to, the group consisting of: radioisotopes, fluorescent compounds, enzymes, and enzyme co-factors. In another embodiment, the label is a label that facilitates purification, isolation, or detection of the polypeptide. The label may be a polyhistidine, FLAG, Glu-Glu, glutathione S transferase (GST), thioredoxin, protein A, protein G, and an immunoglobulin heavy chain constant region. In a preferred embodiment, the labeled protein is TNF-α. In another preferred embodiment, the labeled protein is TNFR.

In an embodiment, the present invention contemplates protein complexes comprising fusion protein(s), wherein said fusion protein comprises a domain that facilitates purification, isolation, or detection of said fusion protein. The fusion domain may be selected from, for example, the group consisting of: polyhistidine, FLAG, Glu-Glu, glutathione S transferase (GST), thioredoxin, protein A, protein G, and an immunoglobulin heavy chain constant region. A preferred fusion domain is FLAG. In certain embodiments, the complex comprises a TNF-α fusion protein. In certain embodiments, the complex comprises a TNFR fusion protein. In certain embodiments, the complex comprises a TNF-α fusion protein and a TNFR fusion protein

As noted above, in certain embodiments, protein complexes of the present invention comprise at least one fragment of any polypeptide component in the complex. In certain embodiments, the complex comprises a fragment of a TNF-α polypeptide, a full-length TNFR polypeptide, and a full-length polypeptide selected from the group consisting of: NAK, RasGAP3, TRCP1, and TRCP2. In certain embodiments, the complex comprises a fragment of a TNF-α polypeptide, a fragment of a TNFR polypeptide, and a full-length polypeptide selected from the group consisting of: NAK, RasGAP3, TRCP1, and TRCP2. In certain embodiments, the complex comprises a full-length TNF-α polypeptide, a fragment of a TNFR polypeptide, and a fragment of a polypeptide selected from the group consisting of: NAK, RasGAP3, TRCP1, and TRCP2. In certain embodiments, the complex comprises a full-length TNF-α polypeptide, a full-length TNFR polypeptide, and a fragment of a polypeptide selected from the group consisting of: NAK, RasGAP3, TRCP1, and TRCP2. In certain embodiments, the complex comprises a fragment of TNF-α polypeptide, a full-length of a TNFR polypeptide, and a fragment of a polypeptide selected from the group consisting of: NAK, RasGAP3, TRCP1, and TRCP2. In certain embodiments, the complex comprises a fragment of TNF-α polypeptide, a fragment of a TNFR polypeptide, and a fragment of a polypeptide selected from the group consisting of: NAK, RasGAP3, TRCP1, and TRCP2.

In preferred embodiments, a fragment of any of the preceding polypeptides is a functional fragment that, for example, retains the ability to associate with at least one other polypeptide of the complex. An example of a functional fragment of a TNFR1 is a fragment that encompasses an intracellular death domain (DD) of TNFR1, approximately 80 amino acids towards the carboxyl-end of TNFR1. An additional example of a functional fragment is a fragment that encompasses a Serine/Treonine protein kinase catalytic domain (S_TKc) of NAK, approximately 235 amino acids at the amino-end of NAK. In certain embodiments, a fragment in the complex encompasses a domain of RasGAP3 selected from the group consisting of: 1) a protein kinase C conserved region 2 domain (C2), approximately 100 amino acids at the amino-terminus; 2) a RasGAP domain, approximately 320 amino acids in the center region; 3) a pleckstrin homology domain (PH), approximately 100 amino acids towards the carboxyl-end; 4) a BTK domain, approximately 35 amino acids towards the carboxyl-end.

In certain embodiment, a complex of the invention is in the water-soluble form (a “soluble complex”). For example, a soluble may comprise a soluble cytoplasmic portion of a TNFR and at least one polypeptide selected from the group consisting of: NAK, RasGAP3, TRCP1, and TRCP2. Optionally, such protein complexes further comprise at least one polypeptide selected from the group consisting of: TRADD, TRAF2, and TRAP2 or a protein known to form a complex with TNFR, such as TRAF1, RIP, RAIDD, or FADD. In certain embodiments, the complex is in a water-insoluble or membrane-associated form. For example, a complex comprising a protein having a transmembrane domain (such as a full-length TNFR) will generally be water-insoluble. Insoluble complexes may be prepared, for example, as lipid micelles, detergent micelles or mixed micelles comprising lipids, detergents and/or other components. Insoluble complexes may also be prepared as membrane fractions from a cell. A membrane fraction may be a crude membrane fraction, wherein the membrane portion is simply separated from the soluble portion of a cell by, for example, centrifugation or filtration. A membrane fraction may be further purified by, for example, affinity purification directed to an affinity tag present in one or more of the proteins of a complex. Where a complex is present in a lipid bilayer, the lipid bilayer may, for example, be a vesicle (optionally inverted, i.e., with the normally extracellular face facing inwards towards the interior of the vesicle) or a planar bilayer. In embodiments where a complexes comprises a TNFR, the TNFR is preferably TNFR1 or a variant thereof (e.g., a soluble cytoplasmic portion, a DD domain, etc.).

In certain embodiments, the present invention also provides additional methods of producing a functional protein complex. In a preferred embodiment, such methods comprise (i) transfecting a cell with a polynucleotide encoding a polypeptide selected from the group consisting of: NAK, RasGAP3, TRCP1, and TRCP2; (ii) contacting said cell with TNF-α polypeptide; (iii) thereby forming a protein complex.

IV. Polypeptides of Protein Complexes

In certain aspects, complexes of the invention comprise polypeptides selected from the group consisting of: TNF-α, TNFR, NAK, RasGAP3, TRCP1, TRCP2, TRADD, TRAF2, and TRAP2, and additional components described herein, and variants polypeptides thereof. In certain embodiments, variant polypeptides have an amino acid sequence that is at least 75% identical to an amino acid sequence as set forth in any of SEQ ID Nos: 1, 3, 5, 7, 9, 11, 13, 15, 17, and 19. In other embodiments, the variant polypeptide has an amino acid sequence at least 80%, 85%, 90%, 95%, 97%, 98%, 99% or 100% identical to an amino acid sequence as set forth in any of SEQ ID Nos: 1, 3, 5, 7, 9, 11, 13, 15, 17, and 19. Preferred polypeptides ofthe complex are the native human NAK, RasGAP3, TRCP1, and TRCP2 sequences (SEQ ID Nos: 13, 15, 17, and 19).

In certain aspects, protein complexes comprise variant polypeptides that are agonists or antagonists of polypeptides as set forth in any of SEQ ID Nos: 1, 3, 5, 7, 9, 11, 13, 15, 17, and 19. Variants of these polypeptides may have a hyperactive or constitutive activity, or, alternatively, act to prevent TNF-α-dependent formation of the protein complex. For example, a truncated form lacking one or more domain may have a dominant negative effect.

In certain aspects, protein complexes comprise variant polypeptides derived from a full-length polypeptides as set forth in any of SEQ ID Nos: 1, 3, 5, 7, 9, 11, 13, 15, 17, and 19. Isolated peptidyl portions of these polypeptides can be obtained by screening polypeptides recombinantly produced from the corresponding fragment of the nucleic acid (encoding such polypeptides) as set forth in any of SEQ ID Nos: 2, 4, 6, 8, 10, 12, 14, 16, 18, and 20. In addition, fragments can be chemically synthesized using techniques known in the art such as conventional Merrifield solid phase f-Moc or t-Boc chemistry. For example, any one of the subject proteins can be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of the formation of a TNFR protein complex.

In certain aspects, protein complexes comprise variant polypeptides containing one or more fusion domain(s). Well known examples of such fusion domains include, for example, polyhistidine, Glu-Glu, glutathione S transferase (GST), thioredoxin, protein A, protein G, and an immunoglobulin heavy chain constant region (Fc), maltose binding protein (MBP), which are particularly useful for isolation of the fusion polypeptide by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners.

Another fusion domain well known in the art is green fluorescent protein (GFP). This fusion partner serves as a fluorescent “tag” which allows the fusion polypeptide of the invention to be identified by fluorescence microscopy or by flow cytometry. The GFP tag is useful when assessing subcellular localization of the fusion polypeptide of the invention, or assessing the co-localization of at least two polypeptides of the protein complex of which one polypeptide is fused with GFP. The GFP tag is also useful for isolating cells which express the fusion polypeptide of the invention by flow cytometric methods such a fluorescence activated cell sorting (FACS).

Fusion domains also include “epitope tags,” which are usually short peptide sequences for which a specific antibody is available. Well known epitope tags for which specific monoclonal antibodies are readily available include FLAG, influenza virus haemagglutinin (HA), and c-myc tags.

In some cases, the fusion domains have a protease cleavage site, such as for Factor Xa or Thrombin, which allow the relevant protease to partially digest the fusion polypeptide of the invention and thereby liberate the recombinant polypeptide therefrom. The liberated polypeptide can then be isolated from the fusion partner by subsequent chromatographic separation.

In certain embodiment, recombinant proteins of the complex may be conveniently prepared by a person skilled in the art using standard protocols as for example described in Sambrook, et al., Molecular Cloning. A Laboratory Manual (Cold Spring Harbor Press, 1989); Current Protocols In Molecular Biology Eds. Ausubel et al.; and Current Protocols In Protein Science Eds. Coligan et al.

In certain embodiments, variants of polypeptides of a complex may be generated by making one or more conservative substitution in a native polypeptide sequence. For instance, it is reasonable to expect, for example, that an isolated replacement of a leucine with an isoleucine or valine, an aspartate with a glutamate, a threonine with a serine, or a similar replacement of an amino acid with a structurally related amino acid (i.e., conservative mutations) will not have a major effect on the biological activity of the resulting molecule. Conservative replacements are those that take place within a family of amino acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine (see, for example, Biochemistry, 2nd ed., Ed. by L. Stryer, W.H. Freeman and Co., 1981). Whether a change in the amino acid sequence of a polypeptide results in a functional homolog can be readily determined by assessing the ability of the variant polypeptide to produce a response in cells in a fashion similar to the wild-type protein. For instance, such variant forms of polypeptides as set forth in any of SEQ ID Nos: 1, 3, 5, 7, 9, 11, 13, 15, 17, and 19, can be assessed, e.g., for their ability to bind to another polypeptide in the protein complex. Polypeptides in which more than one replacement has taken place can readily be tested in the same manner.

In certain embodiment, the invention further contemplates a method of generating sets of combinatorial mutants of the subject polypeptides, as well as truncation mutants, and is especially useful for identifying potential variant sequences (e.g., homologs) that are functional in forming the protein complex of the invention. The purpose of screening such combinatorial libraries is to generate, for example, homologs which can act as either agonists or antagonist, or alternatively, which possess novel activities all together. Combinatorially-derived homologs can be generated which have a selective potency relative to a naturally occurring polypeptide. Such proteins, when expressed from recombinant DNA constructs, can be used in the assay protocols described herein. In similar fashion, homologs of the subject polypeptides as set forth in any of SEQ ID Nos: 1, 3, 5, 7, 9, 11, 13, 15, 17, and 19, can be generated by the present combinatorial approach to act as antagonists, in that they are able to interfere with the ability of the corresponding wild-type protein to function in the protein complex of the invention.

Other forms of mutagenesis can be utilized to generate a combinatorial library. For example, homologs of the subject proteins (both agonist and antagonist forms) can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol. Biol. 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol. Biol. 7:32-34). Linker scanning mutagenesis, particularly in a combinatorial setting, is an attractive method for identifying truncated (bioactive) forms of the subject proteins.

A wide range of techniques are known in the art for screening gene products of combinatorial libraries made by point mutations and truncations, and, for screening cDNA libraries for gene products having a certain property. Such techniques will be generally adaptable for rapid screening of the gene libraries generated by the combinatorial mutagenesis of homologs of the subject proteins. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

In certain embodiment, the invention also provides for reduction of polypeptides to generate variants that are mimetics, e.g., peptide or non-peptide compounds, which are able to mimic binding of the authentic protein to another cellular partner. Such mutagenic techniques as described above are also particularly useful for mapping the determinants of a polypeptide which participate in protein-protein interactions involved in, for example, the assembly of protein complexes of the invention. To illustrate, the critical residues of a polypeptide (such as NAK), which are involved in molecular recognition of an interactive protein (such as TNFR or TNF-α) can be determined and used to generate NAK polypeptide-derived peptidomimetics which bind to TNFR, and by inhibiting NAK binding, act to inhibit the assembly or signaling activity of the protein complex. By employing, for example, scanning mutagenesis to map the amino acid residues of a polypeptide which are involved in binding to another polypeptide, peptidomimetic compounds can be generated which mimic those residues involved in binding. For instance, non-hydrolyzable peptide analogs of such residues can be generated using benzodiazepine (e.g., see Freidinger et al., in Peptides: Chemistry and Biology, G. R. Marshall ed., ESCOM Publisher: Leiden, Netherlands, 1988), azepine (e.g., see Huffman et al., in Peptides: Chemistry and Biology, G. R. Marshall ed., ESCOM Publisher: Leiden, Netherlands, 1988), substituted gamma lactam rings (Garvey et al., in Peptides: Chemistry and Biology, G. R. Marshall ed., ESCOM Publisher: Leiden, Netherlands, 1988), keto-methylene pseudopeptides (Ewenson et al., (1986) J. Med. Chem. 29:295; and Ewenson et al., in Peptides: Structure and Function (Proceedings of the 9th American Peptide Symposium) Pierce Chemical Co. Rockland, Ill., 1985), b-turn dipeptide cores (Nagai et al., (1985) Tetrahedron Lett. 26:647; and Sato et al., (1986) J. Chem. Soc. Perkin Trans. 1:1231), and b-aminoalcohols (Gordon et al., (1985) Biochem Biophys Res. Commun. 126:419; and Dann et al., (1986) Biochem Biophys Res. Commun. 134:71).

V. Nucleic Acids

In certain aspects, complexes of the invention comprise polypeptides encoded by nucleic acids selected from the group consisting of: TNF-α, TNFR, NAK, RasGAP3, TRCP1, TRCP2, TRADD, TRAF2, TRAP2, and additional components described herein. Nucleic acids are further understood to include nucleic acids that comprise variants of SEQ ID Nos: 2, 4, 6, 8, 10, 12, 14, 16, 18, and 20. Variant nucleotide sequences include sequences that differ by one or more nucleotide substitutions, additions or deletions, such as allelic variants; and will, therefore, include coding sequences that differ from the nucleotide sequence of the coding sequence e.g., due to the degeneracy of the genetic code. In certain embodiments, variant nucleic acids will also include sequences that will hybridize under highly stringent conditions to a nucleotide sequence of a coding sequence designated in any of SEQ ID Nos: 2, 4, 6, 8, 10, 12, 14, 16, 18, and 20. One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the invention provides nucleic acids which hybridize under low stringency conditions of 6×SSC at room temperature followed by a wash at 2×SSC at room temperature.

Isolated nucleic acids which differ from SEQ ID Nos: 2, 4, 6, 8, 10, 12, 14, 16, 18, and 20 due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this invention.

VI. Host Cells

In one embodiment, the invention provides host cells comprising at least three nucleic acids encoding any three polypeptides of a protein complex of the invention or variants thereof. In one embodiment, the first nucleic acid comprises a recombinant nucleic acid encoding a TNF-α polypeptide, wherein the second nucleic acid comprises a recombinant nucleic acid encoding a TNPR polypeptide and wherein the third nucleic acid comprises a recombinant nucleic acid encoding a polypeptide selected from the group consisting of: NAK, RasGAP3, TRCP1, and TRCP2. In one embodiment, the second nucleic acid encodes a TNFR1 polypeptide. In another embodiment, the second nucleic acid encodes a TNFR2 polypeptide. In one embodiment, the third nucleic acid encodes a NAK polypeptide. In another embodiment, the third nucleic acid encodes a RasGAP3 polypeptide. In another embodiment, the third nucleic acid encodes TRCP1 polypeptide. In another embodiment, the third nucleic acid encodes TRCP2 polypeptide.

In certain aspects, the invention provides host cells comprising at least two recombinant nucleic acids encoding any two polypeptides of a protein complex of the invention or variants thereof. In one embodiment, the first nucleic acid comprises a recombinant nucleic acid encoding a TNFR polypeptide, and wherein the second nucleic acid comprises a recombinant nucleic acid encoding a polypeptide selected from the group consisting of: NAK, RasGAP3, TRCP1, and TRCP2. In one embodiment, the first nucleic acid encodes a TNFR1 polypeptide. In another embodiment, the first nucleic acid encodes a TNFR2 polypeptide. In one embodiment, the second nucleic acid encodes a NAK polypeptide. In another embodiment, the second nucleic acid encodes a RasGAP3 polypeptide. In another embodiment, the second nucleic acid encodes TRCP1 polypeptide. In another embodiment, the second nucleic acid encodes TRCP2 polypeptide.

In further aspects, the invention provides host cells comprising a recombinant nucleic acid encoding TRCP1, TRCP2 or a variant thereof. In some embodiments, host cells may be used, for example, for purifying, making or studying a protein or protein complex. Optionally, host cells may be used, for example, for testing compounds in assay protocols such as those described below.

In certain embodiments, recombinant expression of polypeptides of a complex of the invention may be performed separately, and complexes formed therefrom. In another embodiment, recombinant expression of such polypeptides of a complex of the invention may be performed in the same cell, and complexes formed therefrom.

Suitable host cells for recombinant expression include bacteria such as E. coli., Clostridium sp., Pseudomonas sp., yeast, plant cells, insect cells (such as Sf9) and mammalian cells such as fibroblasts, lymphocytes, U937 cells (or other promonocytic cell lines) and Chinese hamster ovary cells (CHO cells).

For the purpose of host cell expression, the recombinant nucleic acid may be operably linked to one or more regulatory sequences in an expression construct. Regulatory nucleotide sequences will generally be appropriate for the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the invention. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used.

The expression vector may also include a fusion domain (typically provided by the expression vector) so that the recombinant polypeptide of the invention is expressed as a fusion polypeptide with said fusion domain. The main advantage of fusion domains are that they assist identification and/or purification of said fusion polypeptide and also enhance protein expression level and overall yield.

VII. Antibodies and Uses Therefor

Another aspect of the invention pertains to an isolated antibody specifically immunoreactive with an epitope of a polypeptide selected from the group consisting of: TNF-α, TNFR, NAK, RasGAP3, TRCP1, and TRCP2, TRADD, TRAF2, and TRAP2 or a protein known to form a complex with TNF-α, such as TRAF1, RIP, RAIDD, or FADD, wherein said antibody disrupts formation of a complex of the invention. By using immunogens derived from an NAK polypeptide (e.g., based on the cDNA sequences), anti-protein/anti-peptide antisera or monoclonal antibodies can be made by standard protocols (see, for example, Antibodies: A Laboratory Manual ed. by Harlow and Lane (Cold Spring Harbor Press: 1988)).

In one embodiment, antibodies of the invention disrupt the formation of an interaction between TNF-α and NAK. In another embodiment, antibodies of the invention disrupt the formation of an interaction between TNF-α and RasGAP3. In another embodiment, antibodies of the invention disrupt the formation of an interaction between TNF-α and TRCP1. In another embodiment, antibodies of the invention disrupt the formation of an interaction between TNF-α and TRCP2.

In one embodiment, the antibody of the invention is a monoclonal antibody. In another embodiment, the antibody of the invention is a Fab fragment. In one embodiment, the antibody of the invention is labeled with a detectable label.

A mammal, such as a mouse, a hamster or rabbit can be immunized with an immunogenic form of the peptide (e.g., an NAK polypeptide or an antigenic fragment which is capable of eliciting an antibody response, or a fusion protein as described above). Techniques for conferring immunogenicity on a protein or peptide include conjugation to carriers or other techniques well known in the art. An immunogenic portion of a polypeptide can be administered in the presence of adjuvant. The progress of immunization can be monitored by detection of antibody titers in plasma or serum. Standard ELISA or other immunoassays can be used with the immunogen as antigen to assess the levels of antibodies. In a preferred embodiment, the subject antibodies are immunospecific for antigenic determinants of polypeptides set forth in SEQ ID NO:14, 16, 18, and 20.

The present invention contemplates antibodies that are specific for a death domain (DD), and preferably the DD domain is part of a TNFR polypeptide. In a more specific embodiment, the DD domain is the region of approximately 80 amino acids in length towards the carboxyl-end of the TNFR1 as set forth in SEQ ID NO: 3.

Following immunization of an animal with an antigenic preparation of the subject polypeptides, antisera can be obtained and, if desired, polyclonal antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with polypeptides of the present invention and monoclonal antibodies isolated from a culture comprising such hybridoma cells. In one embodiment, an anti-NAK antibody specifically reacts with the protein encoded by a nucleic acid having SEQ ID NO:14.

The term antibody as used herein is intended to include fragments thereof which are also specifically reactive with one of the subject polypeptides. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments.

The antibody of the present invention is further intended to include bispecific, single-chain, and chimeric and humanized molecules having affinity for one of the subject polypeptides, conferred by at least one CDR region of the antibody. In preferred embodiments, the antibody further comprises a label attached thereto and able to be detected (e.g., the label can be a radioisotope, fluorescent compound, enzyme or enzyme co-factor).

VII. Drug Screening Assays

In certain embodiments, the present invention provides assays for identifying test compounds which either inhibit or potentiate the stability of the protein complex of the invention. In one aspect, the assays detect test compounds which inhibit or potentiate interaction of one polypeptide with another polypeptide in the protein complex of the invention.

In certain embodiments, the assays detect test compounds which modulate the signaling activities of the protein complex, such as binding to other cellular components, activating enzymes such as caspases, lipases, kinases, and phosphatases, activating NF-kB transcriptional activity, and the like.

A variety of assay formats will suffice and, in light of the present disclosure, those not expressly described herein will nevertheless be comprehended by one of ordinary skill in the art. Assay formats which approximate such conditions as formation of protein complexes, enzymatic activity, and may be generated in many different forms, and include assays based on cell-free systems, e.g., purified proteins or cell lysates, as well as cell-based assays which utilize intact cells. Simple binding assays can be used to detect compounds that inhibit or potentiate the interaction between one polypeptide and another polypeptide in the complex, or the binding of the complex to a substrate. Compounds to be tested can be produced, for example, by bacteria, yeast or other organisms (e.g., natural products), produced chemically (e.g., small molecules, including peptidomimetics), or produced recombinantly.

In many embodiments, a cell is manipulated after incubation with a candidate compound and assayed for activities of the protein complex of invention. In certain embodiments, bioassays for activities of a protein complex may include apoptosis assays (e.g., caspase and TUNEL assays) and NF-kB activity assays (e.g., NF-kB luciferase or GFP reporter gene assays).

Exemplary apoptosis assays (e.g., caspase and TUNEL assays) may be carried out as described by Chaisson et al., (2002) J. Clin. Invest. 110: 193-202. Cells are incubated with a candidate compound or left untreated. Cell lysates are then incubated with a fluorogenic caspase-3 substrate, DEVD-AMC (Alexis Corp., San Diego, Calif., USA), for 1 hour. Fluorescence is quantitated using a fluorescent plate reader (Packard Instrument Co., Meriden, Conn., USA) at an excitation wavelength of 360 nm and an emission wavelength of 460 nm. For the TUNEL assay, apoptotic nuclei are detected using the POD In Situ Cell Death Detection kit (Roche Diagnostics Inc.) according to the manufacturer's instructions. Positive nuclei were counted in 30 fields (×400) for each slide.

Exemplary NF-kB luciferase or GFP reporter gene assays may be carried out as described by Shona et al., (2002) FEBS Letters. 515: 119-126. Briefly, cells are transfected with an NF-kB-luciferase reporter gene. The transfected cells are then incubated with a candidate compound. Subsequently, NF-kB-stimulated luciferase activity is measured in cells treated with the compound or without the compound. Alternatively, cells can be transfected with an NF-kB-GFP reporter gene (Stratagene). The transfected cells are then incubated with a candidate compound. Subsequently, NF-kB-stimulated gene activity is monitored by measuring GFP expression with a fluorescence/visible light microscope set-up or by FACS analysis.

In certain embodiments, the present invention provides reconstituted protein preparations including a polypeptide of the complex, and one or more interacting polypeptides of the complex. Assays of the present invention include labeled in vitro protein-protein binding assays, immunoassays for protein binding, and the like. The purified protein may also be used for determination of three-dimensional crystal structure, which can be used for modeling intermolecular interactions.

In certain embodiments of the present assays, component polypeptides of the complex can be endogenous to the cell selected to support the assays. Alternatively, some or all of the component polypeptides can be derived from exogenous sources. For instance, fusion proteins can be introduced into the cell by recombinant techniques (such as through the use of an expression vector), as well as by microinjecting the fusion protein itself or mRNA encoding the fusion protein.

In further embodiments of the assays, a complex can be generated in whole cells, taking advantage of cell culture techniques to support the subject assays. For example, as described below, a complex can be constituted in a eukaryotic cell culture system, including mammalian and yeast cells. Advantages to generating the subject assays in an intact cell include the ability to detect compounds which are functional in an environment more closely approximating that which therapeutic use of the compounds would require, including the ability of the compound to gain entry into the cell. Furthermore, certain of the in vivo embodiments of the assay, such as examples given below, are amenable to high through-put analysis of candidate compounds.

In certain in vitro embodiments of the present assay, a reconstituted complex comprises a reconstituted mixture of at least semi-purified proteins. By semi-purified, it is meant that the proteins utilized in the reconstituted mixture have been previously separated from other cellular proteins. For instance, in contrast to cell lysates, proteins involved in the complex formation are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular origin) which might interfere with or otherwise alter the ability to measure the complex assembly and/or disassembly.

In certain embodiments, assaying in the presence and absence of a candidate compound, can be accomplished in any vessel suitable for containing the reactants. Examples include microtitre plates, test tubes, and micro-centrifuge tubes.

In certain embodiments, drug screening assays can be generated which detect test compounds on the basis of their ability to interfere with assembly, stability, or function of a complex of the invention. In an exemplary binding assay, the compound of interest is contacted with a mixture comprising a TNF-α polypeptide, a TNFR polypeptide and at least one polypeptide selected from the group consisting of: NAK, RasGAP3, TRCP1, and TRCP2. Detection and quantification of the complex provide a means for determining the compound's efficacy at inhibiting (or potentiating) interaction between the two component polypeptides. The efficacy of the compound can be assessed by generating dose response curves from data obtained using various concentrations of the test compound. Moreover, a control assay can also be performed to provide a baseline for comparison. In the control assay, the formation of complexes is quantitated in the absence of the test compound.

In certain embodiments, association between any two polypeptides in a complex or between the complex and a substrate polypeptide, may be detected by a variety of techniques, many of which are effectively described above. For instance, modulation in the formation of complexes can be quantitated using, for example, detectably labeled proteins (e.g., radiolabeled, fluorescently labeled, or enzymatically labeled), by immunoassay, or by chromatographic detection. Surface plasmon resonance systems, such as those available from Biacore International AB (Uppsala, Sweden), may also be used to detect protein-protein interaction.

In certain embodiments, one of the polypeptides of a complex can be immobilized to facilitate separation of the complex from uncomplexed forms of one of the polypeptides, as well as to accommodate automation of the assay. In an illustrative embodiment, a fusion protein can be provided which adds a domain that permits the protein to be bound to an insoluble matrix. For example, GST-NAK fusion protein can be adsorbed onto glutathione sepharose beads (Sigma Chemical, St. Louis, Mo.) or glutathione derivatized microtitre plates, which are then combined with a potential interacting protein (e.g., an ³⁵S-labeled TNFR polypeptide), and the test compound are incubated under conditions conducive to complex formation. Following incubation, the beads are washed to remove any unbound interacting protein, and the matrix bead-bound radiolabel determined directly (e.g., beads placed in scintillant), or in the supernatant after the complexes are dissociated, e.g., when microtitre plate is used. Alternatively, after washing away unbound protein, the complexes can be dissociated from the matrix, separated by SDS-PAGE gel, and the level of interacting polypeptide found in the matrix-bound fraction quantitated from the gel using standard electrophoretic techniques.

In a further embodiment, compounds that bind to a complex may be identified by using an immobilized complex. In an illustrative embodiment, a fusion protein of a complex can be provided which adds a domain that permits the complex to be bound to an insoluble matrix. For example, a complex including a component of GST-TNF-α fusion protein can be adsorbed onto glutathione sepharose beads (Sigma Chemical, St. Louis, Mo.) or glutathione derivatized microtitre plates, which are then combined with a potential labeled binding compound and incubated under conditions conducive to binding. Following incubation, the beads are washed to remove any unbound compound, and the matrix bead-bound label determined directly, or in the supernatant after the bound compound is dissociated.

In yet another embodiment, a two-hybrid assay (also referred to as an interaction trap assay) can be used for detecting the interaction of any two polypeptides in the complex (see also, U.S. Pat. No. 5,283,317; Zervos et al., (1993) Cell 72:223-232; Madura et al., (1993) J. Biol. Chem. 268:12046-12054; Bartel et al., (1993) Biotechniques 14:920-924; and Iwabuchi et al,. (1993) Oncogene 8:1693-1696), and for subsequently detecting test compounds which inhibit or potentiate binding of the proteins to one and other. This assay includes providing a host cell, for example, a yeast cell (preferred), a mammalian cell or a bacterial cell type. The host cell contains a reporter gene having a binding site for the DNA-binding domain of a transcriptional activator used in the bait protein, such that the reporter gene expresses a detectable gene product when the gene is transcriptionally activated. A first chimeric gene is provided which is capable of being expressed in the host cell, and encodes a “bait” fusion protein. A second chimeric gene is also provided which is capable of being expressed in the host cell, and encodes the “fish” fusion protein. In one embodiment, both the first and the second chimeric genes are introduced into the host cell in the form of plasmids. Preferably, however, the first chimeric gene is present in a chromosome of the host cell and the second chimeric gene is introduced into the host cell as part of a plasmid.

Preferably, the DNA-binding domain of the first hybrid protein and the transcriptional activation domain of the second hybrid protein are derived from transcriptional activators having separable DNA-binding and transcriptional activation domains. For instance, these separate DNA-binding and transcriptional activation domains are known to be found in the yeast GALA protein, and are known to be found in the yeast GCN4 and ADR1 proteins. Many other proteins involved in transcription also have separable binding and transcriptional activation domains which make them useful for the present invention, and include, for example, the LexA and VP16 proteins. It will be understood that other (substantially) transcriptionally-inert DNA-binding domains may be used in the subject constructs; such as domains of ACE1, 1cI, lac repressor, jun or fos. In another embodiment, the DNA-binding domain and the transcriptional activation domain may be from different proteins. The use of a LexA DNA binding domain provides certain advantages. For example, in yeast, the LexA moiety contains no activation function and has no known effect on transcription of yeast genes. In addition, use of LexA allows control over the sensitivity of the assay to the level of interaction (see, for example, the Brent et al., PCT publication WO94/10300).

In certain embodiments, the invention provides a two-hybrid assay to identify test compounds that inhibit or potentiate the stability of the complex. To illustrate, a first fusion protein (i.e., a “bait” protein) comprising a TNFR polypeptide and a second fusion protein (i.e., a “fish” protein) comprising a polypeptide selected from the group consisting of: NAK, RasGAP3, TRCP1, and TRCP2, are introduced in the host cell. Cells are subjected to conditions under which the bait and fish fusion proteins are expressed in sufficient quantity for the reporter gene to be activated. The interaction of the two fusion polypeptides of the complex results in a detectable signal produced by the expression of the reporter gene. Accordingly, the level of interaction between the two fusion proteins in the presence of a test compound and in the absence of the test compound can be evaluated by detecting the level of expression of the reporter gene in each case. Various reporter constructs may be used in accord with the methods of the invention and include, for example, reporter genes which produce such detectable signals as selected from the group consisting of: an enzymatic signal, a fluorescent signal, a phosphorescent signal and drug resistance.

In many drug screening programs which test libraries of compounds and natural extracts, high throughput assays are desirable in order to maximize the number of compounds surveyed in a given period of time. Assays of the present invention which are performed in cell-free systems, such as may be developed with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

In certain embodiments, activities of a protein complex may include, without limitation, a protein complex formation, which may be assessed by immunoprecipitation and analysis of co-immunoprecipitated proteins or affinity purification and analysis of co-purified proteins. Fluorescence Resonance Energy Transfer (FRET)-based assays may also be used to determine complex formation. Fluorescent molecules having the proper emission and excitation spectra that are brought into close proximity with one another can exhibit FRET. The fluorescent molecules are chosen such that the emission spectrum of one of the molecules (the donor molecule) overlaps with the excitation spectrum of the other molecule (the acceptor molecule). The donor molecule is excited by light of appropriate intensity within the donor's excitation spectrum. The donor then emits the absorbed energy as fluorescent light. The fluorescent energy it produces is quenched by the acceptor molecule. FRET can be manifested as a reduction in the intensity of the fluorescent signal from the donor, reduction in the lifetime of its excited state, and/or re-emission of fluorescent light at the longer wavelengths (lower energies) characteristic of the acceptor. When the fluorescent proteins physically separate, FRET effects are diminished or eliminated. (U.S. Pat. No. 5,981,200).

For example, a cyan fluorescent protein is excited by light at roughly 425-450 nm wavelength and emits light in the range of 450-500 nm. Yellow fluorescent protein is excited by light at roughly 500-525 nm and emits light at 525-500 nm. If these two proteins are placed in solution, the cyan and yellow fluorescence may be separately visualized. However, if these two proteins are forced into close proximity with each other, the fluorescent properties will be altered by FRET. The bluish light emitted by CFP will be absorbed by YFP and re-emitted as yellow light. This means that when the proteins are stimulated with light at wavelength 450 nm, the cyan emitted light is greatly reduced and the yellow light, which is not normally stimulated at this wavelength, is greatly increased. FRET is typically monitored by measuring the spectrum of emitted light in response to stimulation with light in the excitation range of the donor and calculating a ratio between the donor-emitted light and the acceptor-emitted light. When the donor:acceptor emission ratio is high, FRET is not occurring and the two fluorescent proteins are not in close proximity. When the donor:acceptor emission ratio is low, FRET is occurring and the two fluorescent proteins are in close proximity. In this manner, the interaction between a first and second polypeptide may be measured.

The occurrence of FRET also causes the fluorescence lifetime of the donor fluorescent moiety to decrease. This change in fluorescence lifetime can be measured using a technique termed fluorescence lifetime imaging technology (FLIM) (Verveer et al., (2000) Science 290: 1567-1570; Squire et al., (1999) J. Microsc. 193: 36; Verveer et al., (2000) Biophys. J. 78: 2127). Global analysis techniques for analyzing FLIM data have been developed. These algorithms use the understanding that the donor fluorescent moiety exists in only a limited number of states each with a distinct fluorescence lifetime. Quantitative maps of each state can be generated on a pixel-by-pixel basis.

To perform FRET-based assays, a polypeptide of a complex (e.g., TNFR) and the interacting protein of interest (e.g., NAK) are both fluorescently labeled. Suitable fluorescent labels are, in view of this specification, well known in the art. Examples are provided below, but suitable fluorescent labels not specifically discussed are also available to those of skill in the art. Fluorescent labeling may be accomplished by expressing a polypeptide as a fusion protein with a fluorescent protein, for example fluorescent proteins isolated from jellyfish, corals and other coelenterates. Exemplary fluorescent proteins include the many variants of the green fluorescent protein (GFP) of Aequoria Victoria. Variants may be brighter, dimmer, or have different excitation and/or emission spectra. Certain variants are altered such that they no longer appear green, and may appear blue, cyan, yellow or red (termed BFP, CFP, YFP and RFP, respectively). Fluorescent proteins may be stably attached to polypeptides through a variety of covalent and noncovalent linkages, including, for example, peptide bonds (eg. expression as a fusion protein), chemical cross-linking and biotin-streptavidin coupling. For examples of fluorescent proteins, see U.S. Pat. Nos. 5,625,048; 5,777,079; 6,066,476; 6,124,128; Prasher et al. (1992) Gene, 111:229-233; Heim et al. (1994) Proc. Natl. Acad. Sci., USA, 91:12501-04; Ward et al. (1982) Photochem. Photobiol., 35:803-808 ; Levine et al. (1982) Comp. Biochem. Physiol., 72B:77-85; Tersikh et al. (2000) Science 290: 1585-88.

FRET-based assays may be used in cell-based assays and in cell-free assays. FRET-based assays are amenable to high-throughput screening methods including Fluorescence Activated Cell Sorting and fluorescent scanning of microtiter arrays.

In general, where a screening assay is a binding assay (whether protein-protein binding, compound-protein binding, etc.), one or more of the molecules may be joined to a label, where the label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins, e.g., albumin, detergents, etc that are used to facilitate optimal protein-protein binding and/or reduce nonspecific or background interactions. Reagents that improve the efficiency of the assay, such as protease inhibitors, nuclease inhibitors, anti- microbial compounds, etc. may be used. The mixture of components are added in any order that provides for the requisite binding. Incubations are performed at any suitable temperature, typically between 4° and 40° C. Incubation periods are selected for optimum activity, but may also be optimized to facilitate rapid high-throughput screening.

In certain embodiments, the invention provides complex-independent assays that are directed to a single polypeptide of the complex, such as TRCP1 or TRCP2. Such assays comprise identifying a test compound that is a candidate modulator of inflammation or apoptosis.

In an exemplary embodiment, a compound that bind to a TRCP1 or TRCP2 may be identified by using an immobilized TRCP1 or TRCP2 polypeptide. In an illustrative embodiment, a fusion protein of a TRCP1 or TRCP2 can be provided which adds a domain that permits the protein to be bound to an insoluble matrix. For example, a TRCP1 or TRCP2 fused with a GST protein can be adsorbed onto glutathione sepharose beads (Sigma Chemical, St. Louis, Mo.) or glutathione derivatized microtitre plates, which are then combined with a potential labeled binding compound and incubated under conditions conducive to binding. Following incubation, the beads are washed to remove any unbound compound, and the matrix bead-bound label determined directly, or in the supernatant after the bound compound is dissociated.

In certain embodiments, a label can directly or indirectly provide a detectable signal. Various labels include radioisotopes, fluorescers, chemiluminescers, enzymes, specific binding molecules, particles, e.g., magnetic particles, and the lice. Specific binding molecules include pairs, such as biotin and streptavidin, digoxin and antidigoxin etc. For the specific binding members, the complementary member would normally be labeled with a molecule that provides for detection, in accordance with known procedures. In certain embodiments, such methods comprise forming the mixture in vitro. In certain embodiments, such methods comprise cell-based assays by forming the mixture in vivo. In certain embodiments, the methods comprise contacting a cell that expresses a TRCP1 or TRCP2 polypeptide or a variant thereof with the test compound.

In certain embodiments, assays are based on cell-free systems, e.g., purified proteins or cell lysates, as well as cell-based assays which utilize intact cells. Simple binding assays can be used to detect compounds that interact with the TRCP1 or TRCP2 polypeptide. Compounds to be tested can be produced, for example, by bacteria, yeast or other organisms (e.g., natural products), produced chemically (e.g., small molecules, including peptidomimetics), or produced recombinantly.

Optionally, test compounds identified from these assays may be used to treat TNF-α related diseases.

IX. Methods of Treatment

In certain embodiments, the present invention relates to methods for treating TNF-α related diseases using the protein complex. These methods are particularly aimed at therapeutic treatments of mammals, and more particularly, humans.

In certain aspects, a method for treating a TNF-α-related disease comprises administering an effective amount of an compound that inhibits the interaction of TNF-α or TNFR with a polypeptide selected from the group consisting of: NAK, RasGAP3, TRCP1, and TRCP2.

Preferably, such methods include administration of a small molecule, an antibody, or a peptide, as defined herein.

In certain aspects, a method of treating a TNF-α-related disease which includes an inflammatory or apoptotic component comprises administering an effective amount of a therapeutic composition that modulates TRCP1.

In certain aspects, a method of treating a TNF-α-related disease which includes an inflammatory or apoptotic component comprises administering an effective amount of a therapeutic composition that modulates TRCP2.

Gene therapy is also applicable in this regard with the use of nucleic acids encoding polypeptides of the protein complex, preferably nucleic acids encoding NAK, RasGAP3, TRCP1, and IRCP2 polypeptides.

As used herein, the term “TNF-α related disease refers to any disease that is mediated by TNF-α (and preferably through a TNFR). Exemplary TNF-α related diseases that may be treated in this way include diseases having an inflammatory or an apoptotic component. It is known that TNF-α is involved in apoptotic cell death, cellular proliferation, differentiation, inflammation, tumorigenesis, viral replication, viral infections, bacterial infections, parasitic infenctions, immune disorders, autoimmune pathologies, graft-versus-host pathologies. A few examples of TNF-α related disorders include cancer, rheumatoid arthritis, Chron's disease, asthma, septic shock, irritable bowel disorder, haemorrhagic fever, and cachexia, the tissue wasting disorder often seen in cancer patients (see, for example, MacEwan D J., (2002) Cellular Signaling, 14:477-492).

Exemplification

The invention now being generally described, it will be more readily understood by reference to the following examples, which are included merely for purposes of illustration of certain aspects and embodiments of the present invention, and are not intended to limit the invention.

### EXAMPLE 1

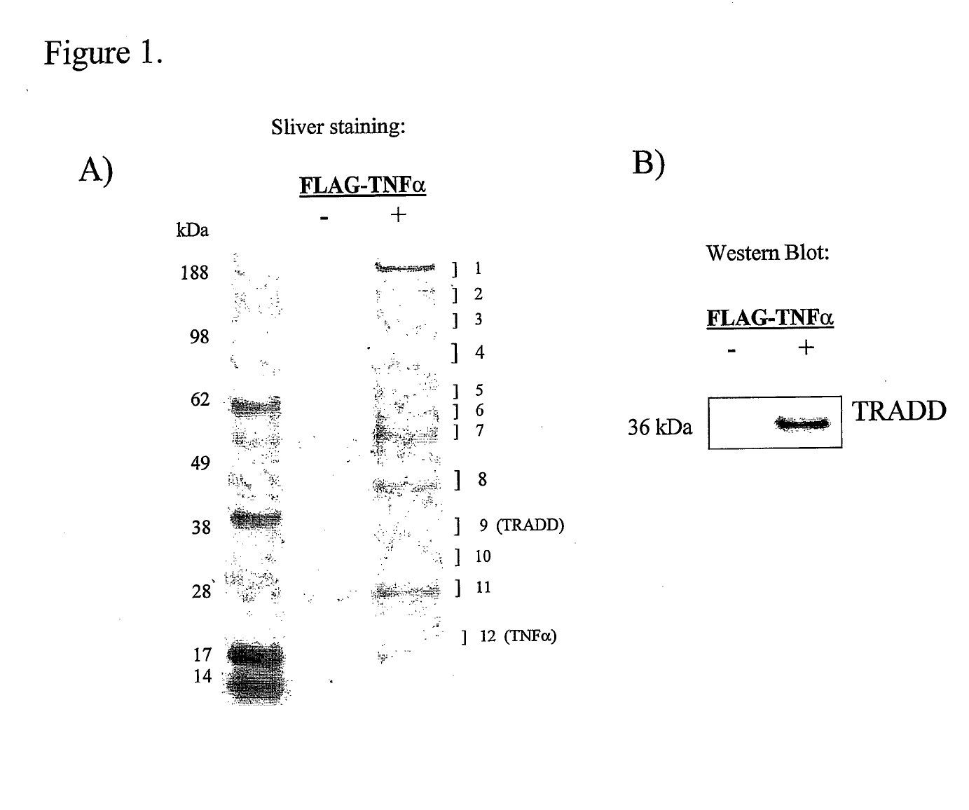

This exemplification describes the identification of proteins in the TNF-α receptor complex by using proteomic approaches as show in FIG. 1.

Myelomonocytic leukemia cells (U937 cells) were incubated with or without FLAG-tagged TNF-α. Cells were then lysed, and the cell lysates were immunoprecipitated with an anti-FLAG antibody (M2) conjugated to the Sepharose beads. The immunoprecipitated complex was washed, and then eluted with FLAG peptides. To increase the eluted protein yields, the beads were further eluted with 8 M urea. The eluted proteins were separated by 4-12% SDS-PAGE gels and visualized by silver staining. Protein bands which showed increased level in the TNF-α treated cells compared to the non-treated cells, were excised from the gel. The gel slices were then digested with trypsin and the resultant tryptic peptides were separated on a microcapillary reversed phase column and eluted directly into a Finnigan LcQ ion trap mass spectrometer. Peptides were subjected to further fragmentation in the ion trap, and spectra were collected. The sequences of the spectra were obtained by a database searching using the SEQUEST software. Several proteins were identified in the TNF-α receptor complex by this method, for example TNF-α, TNF-α receptors, TRADD, TRAF2, TRAP2, NAK, RasGAP3, TRCP1, and TRCP2. Although TNF-α receptors are known to associate with such proteins as TRADD, TRAF2 or TRAP2 was known, the association of TNF-α receptors with NAK, RasGAP3, TRCP1, or TRCP2 was not described before. The presence of TRADD in the TNF-α receptor complex was further confirmed by Western Blotting with an anti-TRADD antibody.

### EXAMPLE 2

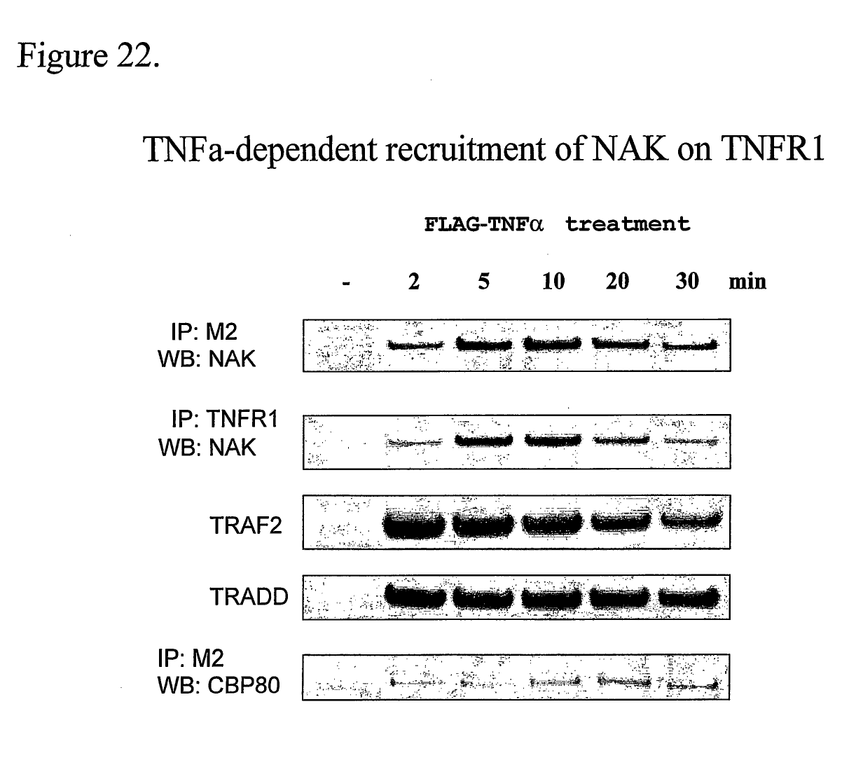

This exemplification describes the ligand-induced binding of the TNFR1 with NAK, TRAF2, or TRADD in a time-dependent manner as shown in FIG. 22.

Myelomonocytic leukemia cells (U937 cells) were incubated with FLAG-tagged TNF-α for 2, 5, 10, 20, or 30 minutes or left untreated. Cells were lysed by 0.5% Triton and the cell lysates were immunoprecipitated with an anti-FLAG antibody (M2) conjugated to the Sepharose beads. The immunoprecipitated complex was washed, eluted with protein sample buffer, and then resolved on 4-12% SDS-PAGE gels. The presence of NAK in the TNF-α receptor complex was detected by western blotting. Similarly, cells lysates were immunoprecipitated with an anti-TNFR1 antibody. The association of the TNF-α receptor with NAK, TRAF2, or TRADD was subsequently detected by western blotting with antibodies against NAK, TRAF2, or TRADD.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. An isolated, purified, or recombinant protein complex comprising:
(i) a tumor necrosis factor alpha (TNF-α) polypeptide or a functional variant thereof;
(ii) a TNF-α receptor (TNFR) polypeptide or a functional variant thereof; and
(iii) at least one polypeptide selected from the group consisting of: NF-κB activating kinase (NAK), RasGAP3, TRCP1, TRCP2 and a functional variant thereof.

2. The complex of claim 1, wherein the TNFR polypeptide is a TNFR1 or TNFR2 polypeptide.

3. The complex of claim 1, comprising a TNF-α polypeptide, a TNFR polypeptide and a NAK polypeptide.

4. The complex of claim 1, comprising a TNF-α polypeptide, a TNFR polypeptide and a RasGAP3 polypeptide.

5. The complex of claim 1, comprising a TNF-α polypeptide, a TNFR polypeptide and a TRCP1 polypeptide.

6. The complex of claim 1, comprising a TNF-α polypeptide, a TNFR polypeptide and a TRCP2 polypeptide.

7. The complex of claim 1, comprising a TNF-α polypeptide, a NAK polypeptide and a TNFR1 polypeptide.

8. The complex of claim 1, further comprising at least one polypeptide selected from the group consisting of: TRADD, TRAF2, TRAP2 and a functional variant thereof.

9. The complex of claim 8, comprising a TNF-α polypeptide, a NAK polypeptide, a TNFR1 polypeptide, a TRAF2 polypeptide and a TRADD polypeptide.

10. The complex of claim 8, comprising a TNF-α polypeptide, a TNFR polypeptide, a NAK polypeptide, a RasGAP3 polypeptide, a TRCP1 polypeptide, a TRCP2 polypeptide, a TRADD polypeptide, a TRAF2-polypeptide, and a TRAP2 polypeptide.

11. The complex of claim 1, wherein said TNF-α is a fusion protein.

12. The complex of claim 1, wherein said TNFR is a fusion protein.

13-16. (canceled)

17. An isolated, purified, or recombinant protein complex comprising:
(i) a TNF-α receptor (TNFR) polypeptide or a functional variant thereof; and
(ii) at least one polypeptide selected from the group consisting of: NF-κB activating kinase (NAK), RasGAP3, TRCP1, TRCP2 and a functional variant thereof.

18-21. (canceled)

22. The complex of claim 17, wherein said TNFR polypeptide is a TNFR1 polypeptide or a TNFR2 polypeptide.

23. The complex of claim 17, further comprising at least one polypeptide selected from the group consisting of: TNF-α, TRADD, TRAF2, and TRAP2.

24. The complex of claim 23, comprising a TNF-α polypeptide, a TNFR polypeptide, a NAK polypeptide, a RasGAP3 polypeptide, a TRCP1 polypeptide, a TRCP2 polypeptide, a TRADD polypeptide, a TRAF2 polypeptide, and a TRAP2 polypeptide.

25. The complex of claim 17, wherein said TNFR polypeptide is a fusion protein.

26-32. (canceled)

33. A host cell comprising a first nucleic acid, a second nucleic acid and a third nucleic acid, wherein the first nucleic acid comprises a recombinant nucleic acid encoding a TNF-α polypeptide, wherein the second nucleic acid comprises a recombinant nucleic acid encoding a TNFR polypeptide and wherein the third nucleic acid comprises a recombinant nucleic acid encoding a polypeptide selected from the group consisting of: NAK, RasGAP3, TRCP1, and TRCP2.

34. The host cell of claim 33, wherein the first nucleic acid comprises a recombinant nucleic acid encoding a TNF-α polypeptide, wherein the second nucleic acid comprises a recombinant nucleic acid encoding a TNFR1 polypeptide and wherein the third nucleic acid comprises a recombinant nucleic acid encoding a NAK polypeptide.

35. A host cell comprising a first nucleic acid and a second nucleic acid, wherein the first nucleic acid comprises a recombinant nucleic acid encoding a TNFR, and wherein the second nucleic acid comprises a recombinant nucleic acid encoding a polypeptide selected from the group consisting of: NAK, RasGAP3, TRCP1, and TRCP2.

36. The host cell of claim 35, wherein the first nucleic acid comprises a recombinant nucleic acid encoding a TNFR1 polypeptide and wherein the second nucleic acid comprises a recombinant nucleic acid encoding a NAK polypeptide.

37. An assay for identifying a test compound which inhibits or potentiates the stability of a complex, comprising:
(a) forming a reaction mixture including:
(i) a TNF-α polypeptide;
(ii) a TNFR polypeptide;
(iii) at least one polypeptide selected from the group consisting of: NAK, RasGAP3, TRCP1, and TRCP2; and
(iv) a test compound; and
(b) detecting the presence of TNF-α or TNFR in the complex;
wherein a change in the presence of TNF-α or TNFR in the complex in the presence of the test compound, relative to the presence of TNF-α or TNFR in the complex in the absence of the test compound, indicates that said test compound potentiates or inhibits the stability of said complex.

38. (canceled)

39. An assay for identifying a test compound which inhibits or potentiates the stability of a complex, comprising:
(a) forming a reaction mixture including:
(i) a TNFR polypeptide;
(ii) at least one polypeptide selected from the group consisting of: NAK, RasGAP3, TRCP1, and TRCP2; and
(iii) a test compound; and
(b) detecting the association between the TNFR and a polypeptide selected from the group consisting of: NAK, RasGAP3, TRCP1, and TRCP2;
wherein a change in the association between TNFR and a polypeptide selected from the group consisting of: NAK, RasGAP3, TRCP1, and TRCP2 in the presence of the test compound, relative to the association between TNFR and a polypeptide selected from the group consisting of: NAK, RasGAP3, TRCP1, and TRCP2 in the absence of the test compound, indicates that said test compound potentiates or inhibits the stability of said complex.

40-43. (canceled)

44. A method for modulating, in a cell, a protein complex comprising at least a first protein and a second protein, wherein said first protein is TNFR, and wherein said second protein is selected from the group consisting of: NAK, RasGAP3, TRCP1, and TRCP2, said method comprising: administering to said cell a compound capable of modulating said protein complex.

45. The method of claim 44, wherein the protein complex further comprises TNF-α.

46. A method of producing a functional complex comprising:
(i) transfecting a cell with a polynucleotide encoding a polypeptide selected from the group consisting of: NAK, RasGAP3, TRCP1, and TRCP2;
(ii) contacting said cell with a TNF-α polypeptide;
(iii) thereby forming a complex.

47. The method of claim 46, further comprising a TNFR polypeptide.

48. A method for treating a TNF-α-related disorder, by administering an effective amount of a compound that inhibits the interaction of TNF-α or TNFR with a polypeptide selected from the group consisting of: NAK, RasGAP3, TRCP1, and TRCP2.

49. The method of claim 48, wherein said compound is selected from the group consisting of: a small molecule, an antibody, and a peptide.

50. A method of identifying a test compound that is a candidate modulator of inflammation or apoptosis, the method comprising:
(i) forming a mixture comprising a TRCP1 polypeptide or a variant polypeptide thereof, and a test compound; and
(ii) measuring the interaction between the TRCP1 polypeptide or the variant and the test compound;
wherein a test compound that interacts with the TRCP1 polypeptide or functional variant is a candidate modulator of inflammation or apoptosis.

51. The method of claim 50, wherein (i) comprises forming the mixture in vitro.

52. The method of claim 50, wherein (i) comprises contacting a cell expressing a TRCP1 polypeptide or a variant thereof, with the test compound.

53. A method of identifying a test compound that is a candidate modulator of inflammation or apoptosis, the method comprising:
(i) forming a mixture comprising a TRCP2 polypeptide or a variant polypeptide thereof, and a test compound; and
(ii) measuring the interaction between the TRCP2 polypeptide or the variant and the test compound;
wherein a test compound that interacts with the TRCP2 polypeptide or functional variant is a candidate modulator of inflammation or apoptosis.

54. The method of claim 53, wherein (i) comprises forming the mixture in vitro.

55. The method of claim 53, wherein (i) comprises contacting a cell expressing a TRCP2 polypeptide or a variant thereof, with the test compound.

56. A method of treating a TNF-α-related disease which includes an inflammatory or apoptotic component, by administering an effective amount of a therapeutic composition that modulates TRCP1 or TRCP2.

57. (canceled)


SEQUENCE LISTING (20 sequences)

SEQ ID NO: 1 - protein, 233 aa, Homo sapiens [sequence omitted]

SEQ ID NO: 2 - DNA, 701 bp, Homo sapiens
atgagcactgaaagcatgatccgggacgtggagctggccgaggaggcgctccccaagaagacaggggggcccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtga

SEQ ID NO: 3 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 4 - DNA, 1367 bp, Homo sapiens
atgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcgtctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatcccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatga

SEQ ID NO: 5 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 6 - DNA, 1384 bp, Homo sapiens
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggcccaggggcagtacacttaccccagccagtgtccacacgatcccaacacacgcagccaactccagaacccagcactgctccaagcacctccttcctgctcccaatgggccccagcccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaa

SEQ ID NO: 7 - protein, 328 aa, Homo sapiens
LAGVGTQAPPRRPGGEMAAGQNGHEEWVGSAYLFVESSLDKVVLSDAYAHPQQKVAVYRALQAALAESGGSPDVLQMLKIHRSDPQLIVQLRFCGRQPCGRFLRAYREGALRAALQRSLAAALAQHSVPLQLELRAGAERLDALLADEERCLSCILAQQPDRLRDEELAELEDALRNLKCGSGARGGDGEVASAPLQPPVPSLSEVKPPPPPPPAQTFLFQGQPVVNRPLSLKDQQTFARSVGLKWRKVGRSLQRGCRALRDPALDSLAYEYEREGLYEQAFQLLRRFVQAEGRRATLQRLVEALEENELTSLAEDLLGLTDPNGGLA

SEQ ID NO: 8 - DNA, 987 bp, Homo sapiens
ctggcgggcgtgggaacccaggccccgccgaggcggccaggaggtgagatggcagctgggcaaaatgggcacgaagagtgggtgggcagcgcatacctgtttgtggagtcctcgctggacaaggtggtcctgtcggatgcctacgcgcacccccagcagaaggtggcagtgtacagggctctgcaggctgccttggcagagagcggcgggagcccggacgtgctgcagatgctgaagatccaccgcagcgacccgcagctgatcgtgcagctgcgattctgcgggcggcagccctgtggccgcttcctccgcgcctaccgcgagggggcgctgcgcgccgcgctgcagaggagcctggcggccgcgctcgcccagcactcggtgccgctgcaactggagctgcgcgccggcgccgagcggctggacgctttgctggcggacgaggagcgctgtttgagttgcatcctagcccagcagcccgaccggctccgggatgaagaactggctgagctggaggatgcgctgcgaaatctgaagtgcggctcgggggcccggggtggcgacggggaggtcgcttcggcccccttgcagcccccggtgccctctctgtcggaggtgaagccgccgccgccgccgccacctgcccagacttttctgttccagggtcagcctgtagtgaatcggccgctgagcctgaaggaccaacagacgttcgcgcgctctgtgggtctcaaatggcgcaaggtggggcgctcactgcagcgaggctgccgggcgctgcgggacccggcgctggactcgctggcctacgagtacgagcgcgagggactgtacgagcaggccttccagctgctgcggcgcttcgtgcaggccgagggccgccgcgccacgctgcagcgcctggtggaggcactcgaggagaacgagctcaccagcctggcagaggacttgctgggcctgaccgatcccaatggcggcctggcctag

SEQ ID NO: 9 - protein, 501 aa, Homo sapiens
MAAASVTSPGSLELLQPGFSKTLLGTRLEAKYLCSACKNILRRPFQAQCGHRYCSFCLTSILSSGPQNCAACVYEGLYEEGISILESSSAFPDNAARREVESLPAVCPNDGCTWKGTLKEYESCHEGLCPFLLTECPACKGLVRLSEKEHHTEQECPKRSLSCQHCRAPCSHVDLEVHYEVCPKFPLTCDGCGKKKIPRETFQDHVRACSKCRVLCRFHTVGCSEMVETENLQDHELQRLREHLALLLSSFLEAQASPGTLNQVGPELLQRCQILEQKIATFENIVCVLNREVERVAVTAEACSRQHRLDQDKIEALSNKVQQLERSIGLKDLAMADLEQKVSELEVSTYDGVFIWKISDFTRKRQEAVAGRTPAIFSPAFYTSRYGYKMCLRVYLNGDGTGRGTHLSLFFVVMKGPNDALLQWPFNQKVTLMLLDHNNREHVIDAFRPDVTSSSFQRPVSDMNIASGCPLFCPVSKMEAKNSYVRDDAIFIKAIVDLTGL

SEQ ID NO: 10 - DNA, 1573 bp, Homo sapiens
atggctgcagctagcgtgaccccccctggctccctggagttgctacagcccggcttctccaagaccctcctggggaccaagctggaagccaagtacctgtgctccgcctgcagaaacgtcctccgcaggcccttccaggcgcagtgtggccaccggtactgctccttctgcctggccagcatcctcagctctgggcctcagaactgtgctgcctgtgttcacgagggcatatatgaagaaggcatttctattttagaaagcagttcggccttcccagataatgctgcccgcagggaggtggagagcctgccggccgtctgtcccagtgatggatgcacctggaaggggaccctgaaagaatacgagagctgccacgaaggccgctgcccgctcatgctgaccgaatgtcccgcgtgtaaaggcctggtccgccttggtgaaaaggagcgccacctggagcacgagtgcccggagagaagcctgagctgccggcattgccgggcaccctgctgcggagcagacgtgaaggcgcaccacgaggtctgccccagttccccttaacttgtgacggctgcggcaagaagaagatcccccgggagaagtttcaggaccacgtcaagttccccttaacttgtgacggctgcggcaagaagaagatcccccgggagaagtttcaggaccacgtcaagacttgtggcaagtgtcgagtcccttgcagattccacgccatcggctgcctcgagacggtagagggtgagaaacagcaggagcacgaggtgcagtggctgcggagcacctggccatgctactgagctcggtgctggaggcaaagcccctcttgggagaccagagccacgcggggtcagagctcctgcagaggtgcgagagcctggagaagaagacggccacttttgagaacattgtctgcgtcctgaaccgggaggtggagagggtggccatgactgccgaggcctgcagccggcagcaccggctggaccaagacaagattgaagccctgagtagcaaggtgcagcagctggagaggagcattggcctcaaggacctggcgatggctgacttggagcagaaggtcaggcccttccaggcgcagtgtggccaccggtactgctccttctgcctggccagcatcctcaggaagctccaggaagctgtggctggccgcatacccgccatcttctccccagccttctacaccagcaggtacggctacaagatgtgtctgcgtatctacctgaacggcgacggcaccgggcgaggaacacacctgtccctcttctttgtggtgatgaagggcccgaatgacgccctgctgcggtggcccttcaaccagaaggtgaccttaatgctgctcgaccagaataaccgggagcacgtgattgacgccttcaggcccgacgtgacttcatcctcttttcagaggccagtcaacgacatgaacatcgcaagcggctgccccctcttctgccccgtctccaagatggaggcaaagaattcctacgtgcgggacgatgccatcttcatcaaggccattgtggacctgacagggctctaa

SEQ ID NO: 11 - protein, 908 aa, Homo sapiens
MEEGGRDKAPVQPQQSPAAAPGGTDEKPSGKERRDAGDKDKEQELSEEDKQLQDELEMLVERLGEKDTSLYRPALEELRRQIRSSTTSMTSVPKPLKFLRPHYGKLKEIYENMAPGENKRFAADIISVLAMTMSGERECLKYRLVGSQEELASWGHEYVRHLAGEVAKEWQELDDAEKVQREPLLTLVKEIVPYNMAHNAEHEACDLLMEIEQVDMLEKDIDENAYAKVCLYLTSCVNYVPEPENSALLRCALGVFRKFSRFPEALRLALMLNDMELVEDIFTSCKDVVVQKQMAFMLGRHGVFLELSEDVEEYEDLTEIMSNVQLNSNFLALARELDIMEPKVPDDIYKTHLENNRFGGSGSQVDSARMNLASSFVNGFVNAAFGQDKLLTDDGNKWLYKNKDHGMLSAAASLGMILLWDVDGGLTQIDKYLYSSEDYIKSGALLACGIVNSGVRNECDPALALLSDYVLHNSNTMRLGSIFGLGLAYAGSNREDVLTLLLPVMGDSKSSMEVAGVTALACGMIAVGSCNGDVTSTILQTIMEKSETELKDTYARWLPLGLGLNHLGKGEAIEAILAALEVVSEPFRSFANTLVDVCAYAGSGNVLKVQQLLHICSEHFDSKEKEEDKDKKEKKDKDKKEAPADMGAHQGVAVLGIALIAMGEEIGAEMALRTFGHLLRYGEPTLRRAVPLALALISVSNPRLNILDTLSKFSHDADPEVSYNSIFAMGMVGSGTNNARLAAMLRQLAQYHAKDPNNLFMVRLAQGLTHLGKGTLTLCPYHSDRQLMSQVAVAGLLTVLVSFLDVRNIILGKSHYVLYGLVAAMQPRMLVTFDEELRPLPVSVRVGQAVDVVGQAGKPKTITGFQTHTTPVLLAHGERAELATEEFLPVTPILEGFVILRKNPNYDL

SEQ ID NO: 12 - DNA, 2727 bp, Homo sapiens
atggaggagggaggccgggacaaggcgccggtgcagccccagcagtctccagcggcggcccccggcggcacggacgagaagccgagcggcaaggagcggcgggatgccggggacaaggacaaagaacaggagctgtctgaagaggataaacagcttcaagatgaactggagatgctcgtggaacgactaggggagaaggatacatccctgtatcgaccagcgctggaggaattgcgaaggcagattcgttcttctacaacttccatgacttcagtgcccaagcctctcaaatttctgcgtccacactatggcaaactgaaggaaatctatgagaacatggcccctggggagaataagcgttttgctgctgacatcatctccgttttggccatgaccatgagtggggagcgtgagtgcctcaagtatcggctagtgggctcccaggaggaattggcatcatggggtcatgagtatgtcaggcatctggcaggagaagtggctaaggagtggcaggagctggatgacgcagagaaggtccagcgggagcctctgctcactctggtgaaggaaatcgtcccctataacatggcccacaatgcagagcatgaggcttgcgacctgcttatggaaattgagcaggtggacatgctggagaaggacattgatgaaaatgcatatgcaaaggtctgcctttatctcaccagttgtgtgaattacgtgcctgagcctgagaactcagccctactgcgttgtgccctgggtgtgttccgaaagtttagccgcttccctgaagctctgagattggcattgatgctcaatgacatggagttggtagaagacatcttcacctcctgcaaggatgtggtagtacagaaacagatggcattcatgctaggccggcatggggtgttcctggagctgagtgaagatgtcgaggagtatgaggacctgacagagatcatgtccaatgtacagctcaacagcaacttcttggccttagctcgggagctggacatcatggagcccaaggtgcctgatgacatctacaaaacccacctagagaacaacaggtttgggggcagtggctctcaggtggactctgcccgcatgaacctggcctcctcttttgtgaatggctttgtgaatgcagcttttggccaagacaagctgctaacagatgatggcaacaaatggctttacaagaacaaggaccacggaatgttgagtgcagctgcatctcttgggatgattctgctgtgggatgtggatggtggcctcacccagattgacaagtacctgtactcctctgaggactacattaagtcaggagctcttcttgcctgtggcatagtgaactctggggtccggaatgagtgtgaccctgctctggcactgctctcagactatgttctccacaacagcaacaccatgagacttggttccatctttgggctaggcttggcttatgctggctcaaatcgtgaagatgtcctaacactgctgctgcctgtgatgggagattcaaagtccagcatggaggtggcaggtgtcacagctttagcctgtggaatgatagcagtagggtcctgcaatggagatgtaacttccactatccttcagaccatcatggagaagtcagagactgagctcaaggatacttatgctcgttggcttcctcttggactgggtctcaaccacctggggaagggtgaggccatcgaggcaatcctggctgcactggaggttgtgtcagagccattccgcagttttgccaacacactggtggatgtgtgtgcatatgcaggctctgggaatgtgctgaaggtgcagcagctgctccacatttgtagcgaacactttgactccaaagagaaggaggaagacaaagacaagaaggaaaagaaagacaaggacaagaaggaagcccctgctgacatgggagcacatcagggagtggctgttctggggattgcccttattgctatgggggaggagattggtgcagagatggcattacgaacctttggccacttgctgagatatggggagcctacactccggagggctgtacctttagcactggccctcatctctgtttcaaatccacgactcaacatcctggataccctaagcaaattctctcatgatgctgatccagaagtttcctataactccatttttgccatgggcatggtgggcagtggtaccaataatgcccgtctggctgcaatgctgcgccagttagctcaatatcatgccaaggacccaaacaacctcttcatggtgcgcttggcacagggcctgacacatttagggaagggcacccttaccctctgcccctaccacagcgaccggcagcttatgagccaggtggccgtggctggactgctcactgtgcttgtctctttcctggatgttcgaaacattattctaggcaaatcacactatgtattgtatgggctggtggctgccatgcagccccgaatgctggttacgtttgatgaggagctgcggccattgccagtgtctgtccgtgtgggccaggcagtggatgtggtgggccaggctggcaagccgaagactatcacagggttccagacgcatacaaccccagtgttgttggcccacggggaacgggcagaattggccactgaggagtttcttcctgttacccccattctggaaggttttgttatccttcggaagaaccccaattatgatctctaa

SEQ ID NO: 13 - protein, 729 aa, Homo sapiens
MQSTSNHLWLLSDILGQGATANVFRGRHKKTGDLFAIKVFNNISFLRPVDVQMREFEVLKKLNHKNIVKLFAIEEETTTRHKVLIMEFCPCGSLYTVLEEPSNAYGLPESEFLIVLRDVVGGMNHLRENGIVHRDIKPGNIMRVIGEDGQSVYKLTDFGAARELEDDEQFVSLYGTEEYLHPDMYERAVLRKDHQKKYGATVDLWSIGVTFYHAATGSLPFRPFEGPRRNKEVMYKIITGKPSGAISGVQKAENGPIDWSGDMPVSCSLSRGLQVLLTPVLANILEADQEKCWGFDQFFAETSDILHRMVIHVFSLQQMTAHKIYIHSYNTATIFHELVYKQTKIISSNQELIYEGRRLVLEPGRLAQHFPKTTEENPIFVVSREPLDTIGLIYEKISLPKVHPRYDLDGDASMAKAITGVVCYACRIASTLLLYQELMRKGIRWLIELIKDDYNETVHKKTEVVITLDFCIRNIEKTVKVYEKLMKINLEAAELGEISDIHTKLLRLSSSQGTIETSLQDIDSRLSPGGSLADAWAHQEGTHPKDRNVEKLQVLLNCMTEIYYQFKKDQAERRLAYNEEQIHKFDKQKLYYHATKAMTHFTDECVKKYEAFLNKSEEWIRKMLHLRKQLLSLTNQCFDIEEEVSKYQEYTNELQETLPQKMFTASSGIKHTMTPIYPSSNTLVEMTLGMKKLKEEMEGVVKELAENNHILERFGSLTMDGGLRNVDCL

SEQ ID NO: 14 - DNA, 2190 bp, Homo sapiens
atgcagagcacttctaatcatctgtggcttttatctgatattttaggccaaggagctactgcaaatgtctttcgtggaagacataagaaaactggtgatttatttgctatcaaagtatttaataacataagcttccttcgtccagtggatgttcaaatgagagaatttgaagtgttgaaaaaactcaatcacaaaaatattgtcaaattatttgctattgaagaggagacaacaacaagacataaagtacttattatggaattttgtccatgtgggagtttatacactgttttagaagaaccttctaatgcctatggactaccagaatctgaattcttaattgttttgcgagatgtggtgggtggaatgaatcatctacgagagaatggtatagtgcaccgtgatatcaagccaggaaatatcatgcgtgttataggggaagatggacagtctgtgtacaaactcacagattttggtgcagctagagaattagaagatgatgagcagtttgtttctctgtatggcacagaagaatatttgcaccctgatatgtatgagagagcagtgctaagaaaagatcatcagaagaaatatggagcaacagttgatctttggagcattggggtaacattttaccatgcagctactggatcactgccatttagaccctttgaagggcctcgtaggaataaagaagtgatgtataaaataattacaggaaagccttctggtgcaatatctggagtacagaaagcagaaaatggaccaattgactggagtggagacatgcctgtttcttgcagtctttctcggggtcttcaggttctacttacccctgttcttgcaaacatccttgaagcagatcaggaaaagtgttggggttttgaccagttttttgcagaaactagtgatatacttcaccgaatggtaattcatgttttttcgctacaacaaatgacagctcataagatttatatacatagctataatactgctactatatttcatgaactggtatataaacaaaccaaaattatttcttcaaatcaagaacttatctacgaagggcgacgcttagtcttagaacctggaaggctggcacaacatttccctaaaactactgaggaaaaccctatatttgtagtaagccgggaacctctggataccataggattaatatatgaaaaaatttccctccctaaagtacatccacgttatgatttagacggggatgctagcatggctaaggcaataacaggggttgtgtgttatgcctgcagaattgccagtaccttactgctttatcaggaattaatgcgaaaggggatacgatggctgattgaattaattaaagatgattacaatgaaactgttcacaaaaagacagaagttgtgatcacattggatttctgtatcagaaacattgaaaaaactgtgaaagtatatgaaaagttgatgaagatcaacctggaagcggcagagttaggtgaaatttcagacatacacaccaaattgttgagactttccagttctcagggaacaatagaaaccagtcttcaggatatcgacagcagattatctccaggtggatcactggcagacgcatgggcacatcaagaaggcactcatccgaaagacagaaatgtagaaaaactacaagtcctgttaaattgcatgacagagatttactatcagttcaaaaaagaccaagcagaacgtagattagcttataatgaagaacaaatccacaaatttgataagcaaaaactgtattaccatgccacaaaagctatgacgcactttacagatgaatgtgttaaaaagtatgaggcatttttgaataagtcagaagaatggataagaaagatgcttcatcttaggaaacagttattatcgctgactaatcagtgttttgatattgaagaagaagtatcaaaatatcaagaatatactaatgagttacaagaaactctgcctcagaaaatgtttacagcttccagtggaatcaaacataccatgaccccaatttatccaagttctaacacattagtagaaatgactcttggtatgaagaaattaaaggaagagatggaaggggtggttaaagaacttgctgaaaataaccacattttagaaaggtttggctctttaaccatggatggtggccttcgcaacgttgactgtctttag

SEQ ID NO: 15 - protein, 834 aa, Homo sapiens
MAVEDEGLRVFQSVKIKIGEAKNLPSYPGPSKMRDCYCTVNLDQEEVFRTKIVEKSLCPFYGEDFYCEIPRSFRHLSFYIFDRDVFRRDSIIGKVAIQKEDLQKYHNRDTWFQLQHVDADSEVQGKVHLELRLSEVITDTGVVCHKLATRIVECQGLPIVNGQCDPYATVTLAGPFRSEAKKTKVKRKTNNPQFDEVFYFEVTRPCSYSKKSHFDFEEEDVDKLEIRVDLWNASNLKFGDEFLGELRIPLKVLRQSSSYEAWYFLQPRDNGSKSLKPDDLGSLRLNVVYTEDHVFSSDYYSPLRDLLLKSADVEPVSASAAHILGEVCREKQEAAVPLVRLFLHYGRVVPFISAIASAEVKRTQDPNTIFRGNSLASKCIDETMKLAGMHYLHVTLKPAIEEICQSHKPCEIDPVKLKDGENLENNMENLRQYVDRVFHAITESGVSCPTVMCDIFFSLREAAAKRFQDDPDVRYTAVSSFIFLRFFAPAILSPNLFQLTPHHTDPQTSRTLTLISKTVQTLGSLSKSKSASFKESYMATFYEFFNEQKYADAVKNFLDLISSSGRRDPKSVEQPIVLKEGFMIKRAQGRKRFGMKNFKKRWFRLTNHEFTYHKSKGDQPLYSIPIENILAVEKLEEESFKMKNMFQVIQPERALYIQANNCVEAKDWIDILTKVSQCNQKRLTVYHPSAYLSGHWLCCRAPSDSAPGCSPCTGGLPANIQLDIDGDRETERIYSLFNLYMSKLEKMQEACGSKSVYDGPEQEEYSTFVIDDPQETYKTLKQVIAGVGALEQEHAQYKRDKFKKTKYGSQEHPIGDKSFQNYIRQQSETSTHSI

SEQ ID NO: 16 - protein, 2505 aa, Homo sapiens
ATGGCGGTGGAGGACGAGGGGCTCCGGGTCTTCCAGAGCGTGAAGATCAAGATCGGTGAAGCCAAAAACCTTCCCTCTTACCCGGGGCCGAGCAAGATGAGGGATTGCTACTGCACGGTGAACCTGGACCAGGAGGAGGTTTTCAGGACCAAAATTGTGGAAAAGTCACTCTGCCCGTTTTACGGAGAAGACTTTTACTGTGAAATTCCTCGGAGCTTTCGTCACCTGTCCTTCTACATTTTCGATAGAGACGTTTTCCGGAGGGATTCCATCATAGGGAAGGTGGCCATCCAGAAGGAGGACTTGCAGAAGTACCACAACAGGGACACCTGGTTCCAGCTGCAGCACGTGGACGCTGACTCGGAAGTGCAGGGCAAAGTGCACCTGGAGCTGCGGCTGAGCGAGGTCATCACAGACACTGGGGTCGTCTGCCACAAGCTCGCCACACGCATCGTCGAGTGCCAGGGCCTCCCCATCGTGAATGGGCAATGTGACCCCTACGCCACCGTGACGCTGGCAGGACCCTTCAGATCAGAAGCAAAGAAGACGAAAGTGAAGAGGAAGACCAACAATCCCCAGTTCGATGAAGTGTTTTATTTTGAGGTGACCCGGCCCTGTAGCTACAGCAAGAAGTCCCACTTTGACTTTGAGGAGGAAGACGTGGACAAGCTCGAAATCAGAGTTGACCTCTGGAATGCCAGTAACCTGAAGTTTGGAGATGAATTCCTGGGAGAACTAAGGATCCCGTTGAAAGTCCTGCGGCAGTCCAGCTCCTACGAGGCGTGGTACTTCCTCCAGCCCCGGGACAATGGTAGCAAGAGCCTAAAGCCAGACGACCTGGGCTCCCTGCGGCTGAACGTGGTATACACGGAAGACCACGTGTTTTCTTCTGACTATTACAGCCCTCTGCGGGACCTGCTGTTGAAGTCTGCGGATGTGGAGCCCGTGTCAGCGTCTGCGGCCCACATCCTGGGCGAGGTTTGCCGGGAGAAGCAGGAGGCGGCCGTCCCGCTGGTGCGGCTCTTCCTACACTATGGCAGGGTGGTGCCATTCATCAGTGCCATCGCCAGCGCGGAGGTGAAGCGGACCCAGGACCCCAACACCATCTTCCGAGGAAACTCACTGGCGTCCAAGTGCATCGACGAGACCATGAAGCTGGCGGGGATGCATTACCTGCATGTCACCCTGAAGCCCGCCATCGAGGAGATATGCCAGAGCCACAAACCCTGTGAAATCGACCCTGTGAAGTTGAAAGACGGAGAAAACCTTGAAAACAACATGGAGAACCTACGGCAGTATGTGGACCGCGTCTTCCACGCCATCACCGAGTCTGGGGTGAGCTGCCCGACCGTCATGTGTGACATCTTCTTCTCCCTCCGGGAGGCGGCGGCCAAGCGCTTCCAGGATGACCCGGACGTCAGGTACACTGCAGTGAGCAGCTTCATCTTCCTGAGGTTCTTTGCGCCCGCCATTCTCTCCCCCAACCTCTTCCAGCTCACGCCGCACCACACGGACCCCCAGACGTCCAGGACGCTGACATTGATCTCCAAGACCGTTCAGACCCTCGGCAGCCTGTCCAAGTCCAAATCTGCGAGTTTTAAGGAGTCCTACATGGCTACATTTTATGAATTCTTCAATGAGCAGAAATATGCTGATGCGGTGAAGAACTTCTTGGATCTGATTTCGTCCTCGGGGAGAAGAGACCCCAAGAGTGTTGAGCAGCCCATCGTGCTTAAAGAAGGGTTCATGATCAAGAGGGCCCAAGGACGGAAGCGCTTTGGGATGAAGAATTTTAAGAAGAGATGGTTTCGCTTGACCAACCATGAATTTACCTACCACAAAAGCAAAGGGGACCAGCCTCTCTACAGCATTCCCATCGAGAACATCCTGGCAGTGGAGAAGCTGGAGGAGGAGTCTTTCAAAATGAAAAACATGTTCCAGGTCATCCAGCCAGAGCGTGCGCTGTACATCCAGGCCAACAACTGCGTGGAGGCCAAGGACTGGATCGACATTCTCACCAAAGTGAGCCAGTGCAACCAGAAGCGCCTCACCGTCTACCACCCGTCCGCCTACCTGAGCGGCCACTGGCTGTGCTGTAGGGCGCCATCCGACTCGGCTCCGGGCTGCTCGCCCTGCACTGGCGGCCTCCCAGCCAACATCCAGCTGGACATTGATGGGGACCGTGAGACGGAGCGTATCTACTCCCTCTTCAACTTGTACATGAGCAAGCTGGAGAAGATGCAGGAGGCCTGTGGGAGCAAATCTGTGTATGACGGCCCGGAGCAGGAGGAGTATTCGACGTTCGTCATTGACGACCCCCAGGAGACCTACAAGACGCTAAAGCAAGTCATCGCTGGGGTTGGGGCTTTGGAGCAGGAGCACGCCCAGTATAAGAGGGACAAGTTCAAGAAGACGAAATATGGAAGCCAGGAGCACCCCATCGGAGACAAGAGCTTCCAGAACTACATCCGGCAGCAGTCCGAGACCTCCACTCATTCCATTTAA

SEQ ID NO: 17 - protein, 821 aa, Homo sapiens
MPTRVCCCCSALRPRYKRLVDNIFPEDPKDGLVKTDMEKLTFYAVSAPEKLDRIGSYLAERLSRDVVRHRSGYVLIAMEALDQLLMACHSQSIKPFVESFLHMVAKLLESGEPKLQVLGTNSFVKFANIEEDTPSYHRRYDFFVSRFSAMCHSCHSDPEIRTEIRIAGIRGIQGVVRKTVNDELRATIWEPQHMDKIVPSLLFNMQKIEEVDSRIGPPSSPSATDKEENPAVLAENCFRELLGRATFGNMNNAVRPVFAHLDHHKLWDPNEFAVHCFKIIMYSIQAQYSHHVIQEILGHLDARKKDAPRVRAGIIQVLLEAVAIAAKGSIGPTVLEVFNTLLKHLRLSVEFEANDLQGGSVGSVNLNTSSKDNDEKIVQNAIIQTIGFFGSNLPDYQRSEIMMFIMGKVPVFGTSTHTLDISQLGDLGTRRIQIMLLRSLLMVTSGYKAKTIVTALPGSFLDPLLSPSLMEDYELRQLVLEVMHNLMDRHDNRAKLRGIRIIPDVADLKIKREKICRQDTSFMKKNGQQLYRHIYLGCKEEDNVQKNYELLYTSLALITIELANEEVVIDLIRLAIALQDSAIINEDNLPMFHRCGIMALVAAYLNFVSQMIAVPAFCQHVSKVIEIRTMEAPYFLPEHIFRDKCMLPKSLEKHEKDLYFLTNKIAESLGGSGYSVERLSVPYVPQVTDEDRLSRRKSIVDTVSIQVDILSNNVPSDDVVSNTEEITFEALKKAIDTSGMEEQEKEKRRLVIEKFQKAPFEEIAAQCESKANLLHDRLAQILELTIRPPPSPSGTLTITSGHAQYQSVPVYEMKFPDLCVY

SEQ ID NO: 18 - DNA, 2466 bp, Homo sapiens
atgcctacccgagtatgctgctgctgttccgctttgcgtcctcgctacaaacgcctggtggacaacatattccctgaagatccaaaagatggccttgtgaaaactgatatggagaaattgacattttatgcagtatctgctccagagaaactggatcgaattggttcttacctggcagaaaggttgagcagggatgttgtcagacatcgttctgggtatgttttgattgctatggaggcactggaccaacttctcatggcttgccattctcaaagcattaagccatttgtagaaagctttcttcatatggtggcaaagctgctggaatcgggggaaccaaagcttcaagttcttggaacaaattcttttgtcaaatttgcaaatattgaagaagacacaccatcctatcacagacgttatgacttttttgtgtctcgattcagtgccatgtgccattcctgtcatagtgatccagaaatacgaacagagatacgaattgctggaattagaggtattcaaggtgtggttcgcaaaacagtcaacgatgaacttcgggccaccatttgggaacctcagcatatggataagattgttccatccctcctgtttaacatgcaaaagatagaagaagttgacagtcgcataggccctccttcttctccttctgcaactgacaaagaagagaatcctgctgtgctggctgaaaactgtttcagagaactgctgggtcgagcaacttttgggaatatgaataatgctgttagaccagtttttgcgcatttagatcatcacaaactgtgggatcccaatgaatttgcagttcactgctttaaaattataatgtattccattcaggctcagtattctcaccatgtgatccaggagattctaggacaccttgatgctcgtaaaaaagatgctccccgggttcgagcaggtattattcaggttctgttagaggctgttgccattgctgctaaaggttccataggtccgacagtgctggaagtcttcaatacccttttgaaacatctgcgtctcagcgttgaattcgaagcaaatgatttacaggggggatctgtaggcagtgtcaacttaaatacaagttccaaagacaatgatgagaagattgtgcagaatgctatcatccaaacaataggattttttggaagtaacctaccagattatcagaggtcagaaatcatgatgttcattatggggaaagtacctgtctttggaacatctacccatactttggatatcagtcaactaggggatttgggaaccaggagaattcagataatgttgctgagatctttgcttatggtgacctctggatataaagcgaagacgattgttactgcactgccagggtctttcctggatcctttgttatcaccatctctcatggaggactacgaactgagacagttggtcttggaagtaatgcataatctcatggatcgtcatgacaatagggcaaagcttcgagggatcagaataataccggatgtagctgacctaaagataaaaagagaaaaaatttgcagacaagacacaagtttcatgaaaaagaatgggcaacagctgtatcggcacatatatttgggttgtaaagaggaagacaacgttcagaaaaactatgaactactttatacttctcttgctcttataactattgaactggctaatgaagaagtagttattgatctcattcgactggccattgctttacaggacagtgcaattatcaatgaggataatttgccaatgttccatcgttgtggaatcatggcactggttgcagcatacctcaactttgtaagtcagatgatagctgtccctgcattttgccagcatgttagcaaggttattgaaattcgaactatggaagccccttattttctaccagagcatatcttcagagataagtgcatgcttccaaaatctttagagaagcatgaaaaagatttgtactttctgaccaacaagattgcagagtcgctaggtggaagtggatatagtgttgagagattgtcagttccgtatgtaccacaagtaacagatgaagatcgactttctagaagaaaaagcattgtggacaccgtatccattcaggtggatattttatccaacaatgttccttctgatgatgtggttagtaacactgaagaaatcacttttgaagcattgaagaaagcaattgataccagtggaatggaagaacaggaaaaggaaaagaggcgtcttgtgatagagaaatttcagaaagcaccttttgaagaaatagcagcacagtgtgaatccaaagcaaatttgcttcatgatagacttgcccaaatattggaactcaccatacgtcctcctcccagtccatcaggaacactgaccattacttctgggcatgcccaataccaatctgtcccagtctatgagatgaagtttccagatctgtgtgtgtactga

SEQ ID NO: 19 - protein, 689 aa, Homo sapiens
MAVVSAVRWLGLRSRLGQPLTGRRAGLCEQARSCRFYSGSATLSKVEGTDVTGIEEVVIPKKKTWDKVAVLQALASTVNRDTTAVPYVFQDDPYLMPASSLESRSFLLAKKSGENVAKFIINSYPKYFQKDIAEPHIPCLMPEYFERQIKDISEAALKERIELRKVKASVDMFDQLLQAGTTVSLETTNSLLDLLCYYGDQEPSTDYHFQQTGQSEALEEENDETSRRKAGHQFGVTWRAKNNAERIFSLMPEKNEHSYCTMIRGMVKHRAYEQALNLYTELLNNRLHADVYTFNALIEATVCAINEKFEEKWSKILELLRHMVAQKVKPNLQTFNTILKCLRRFHVFARSPALQVLREMKAIGIEPSLATYHHIIRLFDQPGDPLKRSSFIIYDIMNELMGKRFSPKDPDDDKFFQSAMSICSSLRDLELAYQVHGLLKTGDNWKFIGPDQHRNFYYSKFFDLICLMEQIDVTLKWYEDLIPSAYFPHSQTMIHLLQALDVANRLEVIPKIWKDSKEYGHTFRSDLREEILMLMARDKHPPELQVAFADCAADIKSAYESQPIRQTAQDWPATSLNCIAILFLRAGRTQEAWKMLGLFRKHNKIPRSELLNELMDSAKVSNSPSQAIEVVELASAFSLPICEGLTQRVMSDFAINQEQKEALSNLTALTSDSDTDSSSDSDSDTSEGK

SEQ ID NO: 20 - DNA, 2068 bp, Homo sapiens
atggcggttgtatctgctgttcgctggctgggcctccgcagcaggcttggccagccgctgacgggtcggcgggcgggtttgtgtgaacaggcacgcagctgcagattttattctggtagtgcaaccctctcaaaggttgaaggaactgatgtaacagggattgaagaagtagtaattccaaaaaagaaaacttgggataaagtagccgttcttcaggcacttgcatccacagtaaacagggataccacagctgtgccttatgtgtttcaagatgatccttaccttatgccagcatcatctttggaatctcgttcatttttactggcaaagaaatccggggagaatgtggccaagtttattattaattcataccccaaatattttcagaaggacatagctgaacctcatataccgtgtttaatgcctgagtactttgaacctcagatcaaagacataagtgaagccgccctgaaggaacgaattgagctcagaaaagtcaaagcctctgtggacatgtttgatcagcttttgcaagcaggaaccactgtgtctcttgaaacaacaaatagtctcttggatttattgtgttactatggtgaccaggagccctcaactgattaccattttcaacaaactggacagtcagagcattggaagaggaaaatgatgagacatctaggaggaaagctggtcatcagtttggagttacatggcgagcaaaaaacaacgctgagagaatcttttctctaatgccagagaaaaatgaacattcctattgcacaatgatccgaggaatggtgaagcaccgagcttatgagcaggcattaaacttgtacactgagttactaaacaacagactccatgctgatgtatacacatttaatgcattgattgaagcaacagtatgtgcgataaatgagaaatttgaggaaaaatggagtaaaatactggagctgctaagacacatggttgcacagaaggtgaaaccaaatcttcagacttttaataccattctgaaatgtctccgaagatttcatgtgtttgcaagatcgccagccttacaggttttacgtgaaatgaaagccattggaatagaaccctcgcttgcaacatatcaccatattattcgcctgtttgatcaacctggagaccctttaaagagatcatccttcatcatttatgatataatgaatgaattaatgggaaagagattttctccaaaggacccggatgatgataagtttttcagtcagccatgagcatatgctcatctctcagagatctagaacttgcctaccaagtacatggccttttaaaaaccggagacaactggaaattcattggacctgatcaacatcgtaatttctattattccaagttcttcgatttgatttgtctaatggaacaaattgatgttaccttgaagtggtatgaggacctgataccttcagcctactttccccactcccaaacaatgatacatcttctccaagcattggatgtggccaatcggctagaagtgattcctaaaatttggaaagatagtaaagaatatggtcatactttccgcagtgacctgagagaagagatcctgatgctcatggcaagggacaagcacccaccagagcttcaggtggcatttgctgactgtgctgctgatatcaaatctgcgtatgaaagccaacccatcagacagactgctcaggattggccagccacctctctcaactgtatagctatcctctttttaagggctgggagaactcaggaagcctggaaaatgttggggcttttcaggaagcataataagattcctagaagtgagttgctgaatgagcttatggacagtgcaaaagtgtctaacagcccttcccaggccattgaagtagtagagctggcaagtgccttcagcttacctatttgtgagggcctcacccagagagtaatgagtgattttgcaatcaaccaggaacaaaaggaagccctaagtaatctaactgcattgaccagtgacagtgatactgacagcagcagtgacagcgacagtgacaccagtgaaggcaaatga